(19) United States
(12) Patent Application Publication
(10) Pub. No.: US 2003/0207400 A1
(43) Pub. Date: Nov. 6, 2003

(54) Vectors for dna delivery

(76) Inventors: Elisabetta Bianchi, Roma (IT); Daniela Fattori, Genzano di Roma (IT); Paolo Ingallinella, Rome (IT); Olaf Kinzel, Roma (IT); Antonello Pessi, Roma (IT)

Correspondence Address: MERCK AND CO INC, P O BOX 2000, RAHWAY, NJ, 070650907

(21) Appl. No.: 10/276,734

(22) PCT Filed: May 11, 2001
(86) PCT No.: PCT/IB01/00980

(51) Int. Cl. C12P 21/02; C12N 5/06; C12N 15/00

(52) U.S. Cl. 435/69.1; 435/320.1; 435/325

(57) ABSTRACT

The present invention pertains to novel products suitable for use as gene delivery systems in which nucleic acid is linked to a ligand in order to facilitate delivery of the nucleic acid to a target cell or sub-cellular compartment via uptake of the ligand. More particularly, the present invention pertains to vectors comprising: (a) a double stranded DNA (dsDNA) having at least one target sequence; and, (b) a chimeric molecule comprising: (i) a sequence specific polyamide (SSP) moiety bound non-covalently to said target sequence; and, (ii) a ligand moiety linked covalently to said sequence specific polyamide. The present invention also pertains to compositions comprising such chimeric molecules and vectors; methods for making such chimeric molecules and vectors; and methods of using such chimeric molecules and vectors, e.g., to deliver nucleic acid vectors to cells or sub-cellular compartments.

### RELATED APPLICATION

This application claims priority to United Kingdom (GB) Patent Application No. 0011938.8, filed May 17, 2000, which is incorporated herein by reference in its entirety.

### TECHNICAL FIELD

The present invention relates to novel products suitable for use as gene delivery systems in which nucleic acid is linked to a ligand in order to facilitate delivery of the nucleic acid to a target cell or sub-cellular compartment via uptake of the ligand.

### BACKGROUND

There are various proposals in the art for chimeric molecules composed of a moiety which binds to the DNA (DNA-binding domain) conjugated to a moiety (e.g., a peptide) which confers additional useful property (functional domain). The functional domain is typically a peptide, such as a nuclear localisation signal, able to confer new properties to the complex with plasmid DNA (pDNA). Examples of the properties conferred by the functional domain are: (i) improved binding to the nuclear transport machinery, hence improved nuclear localization; (ii) binding to a ubiquitous cell-surface receptor, for improved intracellular delivery through receptor-mediated internalization; (iii) binding to a cell/tissue specific receptor, for specific cell/tissue targeting of the plasmid DNA; and, (iv) improved delivery to the nucleus by escape from the endosomal compartment through an endosomolitic peptide. Functional domains which are sugars have also been proposed.

Various DNA binding domains have been proposed, including moieties which bind covalently and other which bind non-covalently. For example, Ciolina, C. et al., 1999, used the SV40 nuclear localisation signal (NLS) attached to a photoactivatable p-azido-tetrafluoro-benzyl group leading to covalent, random binding of the conjugate to plasmid DNA. Similarly, Sebestyen, et al., 1998, used a cyclopropan-pyrroloindole alkylating group attached to the NLS, leading to covalent, random binding of the conjugate to a plasmid DNA. These methods are not usually reproducible in terms of the ratio of peptides attached per kb of DNA, or the site of attachment. Moreover, attachment of conjugates in regions of DNA which need to be transcribed may be deleterious.

There are potentially several advantages of using non-covalent attachment of ligand, provided that the affinity of the DNA binding domain is sufficiently high to ensure functionally permanent conjugation. Polylysine has been proposed for this purpose, though this moiety still has the disadvantage of binding randomly to DNA which is undesirable for the purposes of reproducibility. Hence recently, the only method among those in the literature that appears satisfactory is the use of Peptide Nucleic Acids (PNA) as DNA binding domain. The use of such a domain has been described in Branden et al., 1999 and also proposed in Zelphati et al., 1999.

Complexes of PNA with DNA are formally non-covalent, but the dissociation constants at physiological ionic strength are so low that they behave in practice as covalent complexes. Moreover, while in principle PNA can be addressed to any DNA sequence, the high tendency to aggregate, also by formation of self-complementary duplex structures, puts some practical limitations to their use.

Polyamides based primarily on pyrrole and imidazole units have been found to be capable of sequence specific binding to DNA. Polyamides of this type have been proposed for the purpose of regulation of gene expression by binding to genomic DNA and inhibition of transcription of a target gene; see for example Trauger et al., 1998, Dickinson, et al., 1998, Bremer et al., 1998, Wemmer et al., 1997, Gottesfield et al., 1997, WO98/50582, WO98/50058, WO98/49142, WO98/45248, WO98/37087, WO98/37067, WO9837066, WO98/35702 and WO97/30975, the disclosures of which are incorporated herein by reference.

The general structure of such polyamides consists of two chains, either as a dimer or a single chain hairpin, which binds to the minor groove of DNA. Sequence specificity is determined by a code of oriented side-by-side pairings of N-methylpyrrole (Py) and N-methylimidazole (Im) amino acids. An Im/Py pairing recognises a. G-C base pair, while a Py/Im pairing recognises C-G. A Py/Py pair is degenerate and targets both A-T and T-A, though replacing one pyrrole ring of the Py/Py pair with 3-hydroxypyrrole (denoted Hy, Hp, or Hyp) provides T-A specificity over A-T for the Hy/Py pairing. FIG. 1 summarises this polyamide pairing code. Other modifications to this system are described in the abovementioned references and also herein below.

### SUMMARY OF THE INVENTION

The present invention provides a novel vector (conjugate) which comprises:

(a) a double stranded DNA (dsDNA) having at least one target sequence; and,

(b) a chimeric molecule comprising:

(i) a sequence specific polyamide (SSP) moiety bound non-covalently to said target sequence; and,

(ii) a ligand moiety linked covalently to said sequence specific polyamide moiety.

In a further aspect, the invention provides a chimeric molecule (chimera) comprising:

(i) a sequence specific polyamide (SSP) moiety capable of binding non-covalently to a target nucleic acid sequence; and,

(ii) a ligand moiety linked covalently to said sequence specific polyamide moiety.

Said double stranded DNA may comprise additional sequences, for example a transcribable sequence which is optionally linked to a promoter to provide for expression of said coding sequence.

The invention further provides compositions comprising said conjugates or chimeras; methods of making said conjugates and chimeras; methods of using said conjugates to deliver nucleic acid vectors to cells or sub-cellular compartments; cells so modified; and their progeny.

### BRIEF DESCRIPTION OF THE DRAWINGS

FIG. 1 is a table showing a polyamide pairing code used to target specific DNA sequences.

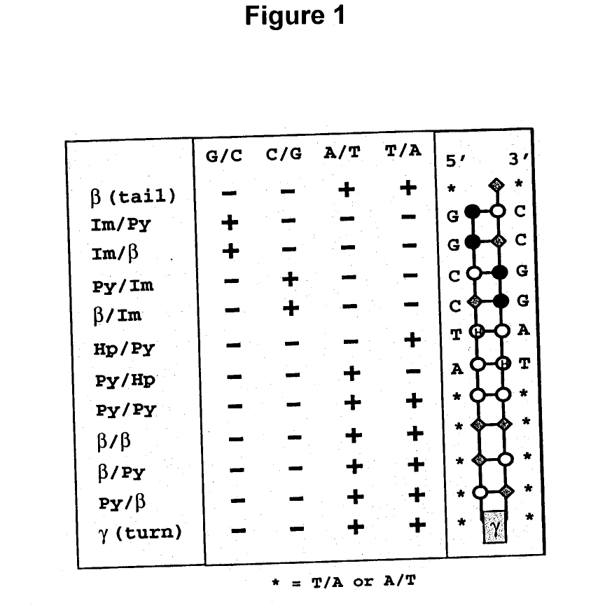

FIG. 2 is a schematic which illustrates DNA binding polyamides.

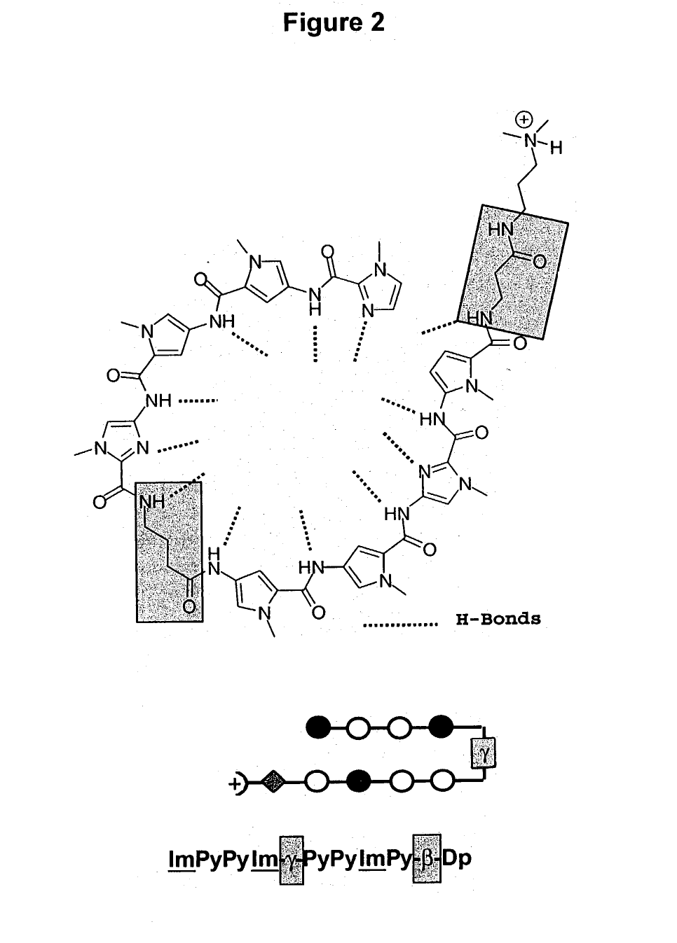

FIG. 3 is a graph of circular dichroism (CD) signal versus wavelength for various concentrations which illustrates that binding to oligonucleotide DNA can be monitored by CD. Upon complexation with double strand DNA, the polyamide becomes chiral.

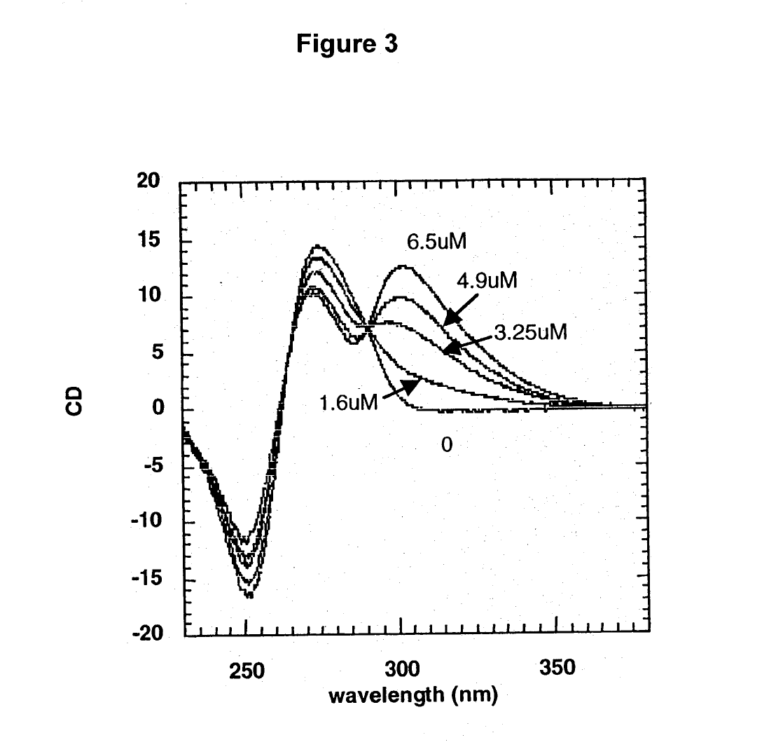

FIG. 4 is a schematic illustrating a polyamide-NLS conjugate for mEPO. The polyamide binding sequence is present twice in the plasmid, as TGCAGCT and AGCAGCA, while the sequence TGCTGCT is present in the EPO gene. The latter can be discriminated against by introduction of a T/A-A/T discriminating Hyp/Py couple instead of the T/A-A/T non-discriminating Py/Py, β/Py, Py/β, and β/β couples.

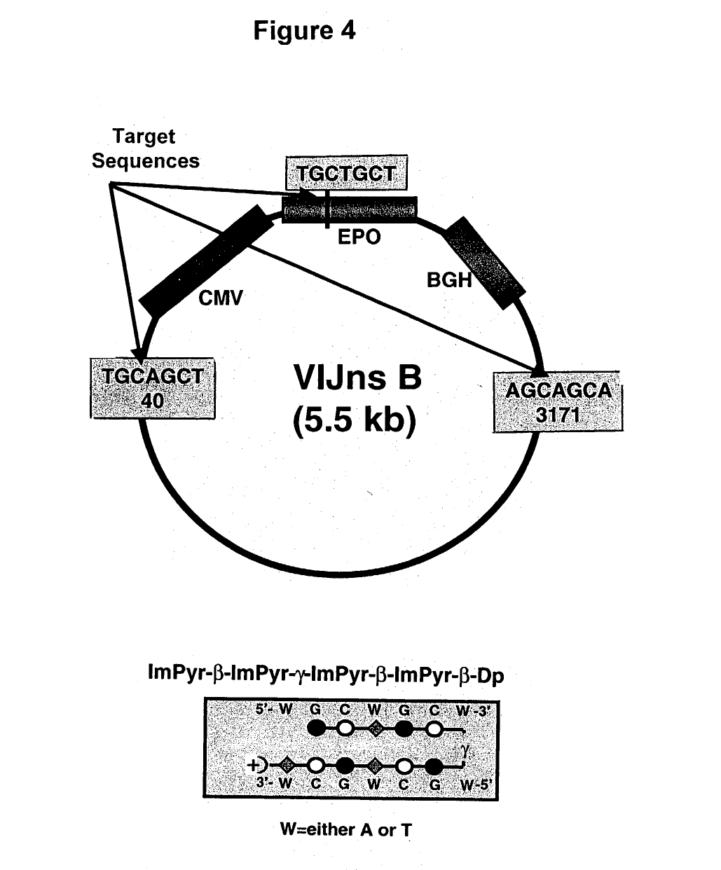

### DETAILED DESCRIPTION OF THE INVENTION

Target Sequence.

The double stranded DNA (dsDNA) element of vectors of the invention will include at least one target sequence and preferably more, for example from 2 to 100, such as from 2 to 10, eg 2, 3, 4, 5 or from 6 to 10 target sequences. The target sequence will be selected to provide a binding region for the sequence specific polyamide.

There are two main criteria in the selection of a target sequence.

It is desirable that the target sequence is not present in other parts of the vector or at the very least the promoter and coding sequence part of the vector. This is in order to ensure that the sequence specific polyamide does not bind to the promoter and coding sequence so as to inhibit the transcription of the coding sequence.

The second main criteria is that the target sequence is of a suitable length to allow non-covalent binding of the polyamide. In order to ensure this, the target sequence will be at least 6 and preferably at least 8 bases in length, for example from 6-20 such as 8-20, or preferably 10-20 bases in length.

Other Elements of the DNA.

The DNA may have further sequences, including dsDNA sequences that can be of human, non-human animal, vegetable, bacterial or viral origin, in particular, sequences found in recombinant viral constructs or in plasmids.

The DNA may include one or more transcribable sequences, promoters, origins of replication and other elements described herein.

An origin of replication may be eukaryotic or prokaryotic, for example in the case of prokaryotic origins to allow for replication of the DNA in a prokaryotic cell in order to facilitate its production or in the case of eukaryotic origins either for replication in culture or for replication in a target host cell.

The DNA may also include one or more selectable or detectable markers. Selectable markers include antibiotic resistance genes. Detectable markers include genes coding for fluorescent or calorimetric proteins such as green fluorescent protein or luciferase.

The DNA may also incorporate sequences designed to facilitate homologous recombination to a specific locus within a host cell, i.e by being homologous to part (e.g., from 150 to 10,000 nucleotides) of that locus.

DNA sequences may be obtained by any method available in the art, such as from genomic or complementary DNA libraries, or by chemical or enzymatic synthesis of sequences known from sequence data banks.

The DNA may be linear or circular, and circular supercoiled molecules are preferred.

Transcribable Sequence.

A transcribable sequence will, when transcribed from the DNA under the control of a promoter, be intended to bring about a therapeutic effect. Generally, such a transcribed sequence will be in the form of mRNA for the expression of a protein. However, it may also be for the production of RNA which itself has a function, for example an anti-sense RNA or a ribozyme.

A wide range of gene therapy vectors have been proposed in the art. These vectors include those with coding sequences designed to modify tumour cells so that the tumour cells may be destroyed or inactivated in some other way. For example, genes encoding enzymes capable of activating pro-drugs into active toxic drugs may be delivered to tumour cells in order to secure localised delivery of toxic compounds to the environment of a tumour. Gene therapy has also been proposed in which tumour suppressor genes, particularly p53, are delivered to tumour cells in order to restore to the cells normal regulation of the cell cycle. Genes encoding cytokines or cell surface markers of the immunoglobulin superfamily may also be delivered to tumour cells in order to enhance the recognition of the cells by the host immune system or to otherwise facilitate an immune response to the cells in the patient.

It is also proposed to deliver genes for therapeutic purposes, for example to provide a functional copy of a gene to an individual subject, particularly a human patient, suffering from a lesion in a gene, either at one allele or more particularly homozygously. For example, it has been proposed to deliver the CTFR gene to the tissues, particularly those of the lungs and the respiratory passages, of patients suffering from cystic fibrosis. Other therapeutic genes include those for blood clotting agents (e.g., factor VIII and factor IX), ADA, alpha-globins, beta-globins and the like. It will be appreciated by those of skill in the art that the precise nature of the coding sequence in the novel conjugates of the invention will be a matter of choice for the skilled person, depending upon the therapeutic application contemplated.

Vectors of the invention may also be used to deliver DNA encoding antigens useful as vaccines. Such antigens may include viral antigens (e.g., antigens from HIV, HSV, CMV, HPV), bacterial antigens (e.g., meningococcal antigens) or host protein antigens to which it is desired to provoke or enhance an immune response, e.g., tumour marker antigens such as CEA.

Promoter.

A wide variety of promoters are known in the art. Promoters include constitutive promoters and tissue specific promoters. Viral promoters may be used as strong constitutive promoters, for example the adenovirus E1A promoter or the CMV promoter. In general, promoters will be selected to be compatible with an intended target host cell and can be selected to be either tissue specific for that cell or constitutive.

Sequence Specific Polyamide (SSP).

The sequence specific polyamide will be based upon pyrrole and imidazole units as described in the references cited above.

Such compounds are oligomers comprising organic cyclic groups joined together by short linkers, which oligomers fit in the minor groove of dsDNA and form complementary pairs with specific nucleotide base pairs in the dsDNA target sequence. These are defined further in WO98/50582. Associated with the organic cyclic compounds are aliphatic amino acids, particularly aliphatic amino acids. In addition, a terminus will desirably have a polar group, conveniently substituted on an alkyl substituent. There will be a consecutive series of at least three complementary pairs of organic heterocycles, where by complementary is intended a preferential juxtaposition with a complementary pair of nucleotides. By appropriate selection of complementary pairs, unpaired organic cyclic compounds in juxtaposition to particular nucleotides of base pairs, aliphatic amino acids, and a polar group substituent, high affinities and high specificities as compared to single-base mismatches can be achieved.

Organic Cyclic Groups.

The oligomers have at least six, more usually at least seven, organic heterocyclic groups (“heterocycles”); eight or more, usually not more than about thirty, more usually not more than about twenty, frequently not more than about eighteen, organic cyclic groups, wherein at least 60%, preferably at least 80%, and more preferably 100% are organic heterocyclic groups.

The heterocycles have five or six annular members (five or six membered rings), particularly five annular members (five membered rings), generally having from one to three, usually one to two annular heteroatoms, where the heteroatoms are selected from nitrogen, oxygen and sulphur, particularly nitrogen, where two heteroatoms are usually spaced apart by at least one intervening carbon atom. The organic cyclic groups are completely unsaturated and will be referred to as aromatic as that term is understood for organic cyclic compounds of from five to six annular members.

Illustrative heterocycles with five annular members include optionally substituted pyrrole, imidazole, pyrazole, triazole, furan, thiophene, oxazole, thiazole, cyclopentadiene, and the like. Illustrative heterocycles with six annular members include optionally substituted pyridine, pyrimidine, triazine, and the like. As indicated below, NH groups in the rings, when substituted, are preferably alkylated with an alkyl group of from one to three carbon atoms, particularly methyl.

The preferred organic cyclic compounds are those with five annular members (five membered rings) having from one to two annular nitrogen atoms (e.g., pyrrole, imidazole, pyrazole), where one of the annular nitrogen atoms is methylated.

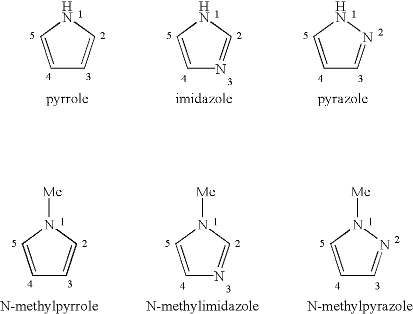

Substituents.

Annular nitrogen atoms may be substituted, depending upon whether the nitrogen atom is directed toward the floor or surface of the groove or away from the groove. Greater latitude in the nature of the substitution is permitted when the nitrogen atom is directed away from the floor of the groove. The orientation of the oligomer is preferably N to C in association with the 5′ to 3′ direction of the strand to which it is juxtaposed.

The heterocycles may be substituted at positions of the heterocycle which are directed away from the floor of the groove for any purpose. Thus, a hydrogen atom may be replaced with a substituent of interest, where the substituent will not result in steric interference with the wall of the minor groove or otherwise create repulsion. When substituted, the substituents may be widely varied, being:

(a) a heteroatom;

(b) a hydrocarbyl of from 1 to 30, more usually 1 to 20, carbon atoms, particularly 1 to 10, more particularly 1 to 6 carbon atoms, including aliphatic, alicyclic, aromatic, and combinations thereof, including both aliphatically saturated and unsaturated, having not more than 10% of the carbon atoms participating in aliphatic unsaturation;

(c) a heterosubstituted hydrocarbyl (where hydrocarbyl is as defined previously), having from 1 to 10, usually 1 to 8, more usually 1 to 6, heteroatoms, including aliphatic, alicyclic, aromatic, and heterocyclic, and combinations thereof, where the heteroatoms are exemplified by halogen, nitrogen, oxygen, sulfur, phosphorous, metal atoms, boron, arsenic, selenium, rare earths, and the like, wherein functional groups are exemplified by amino, including mono- and di-substituted amino, oxy, including hydroxy and oxyether, thio, including mercapto and thioether, oxo, including oxo-carbonyl (aldehyde and ketone) and non-oxo-carbonyl (carboxy, including acyl halide, anhydride, ester, and amide), phosphorous, including phosphines, phosphites, phosphates, phosphoramidites, etc., boron, including borates, boronic acids and boronates, nitro, cyano, azo, azoxy, hydrazino, etc.

The functional groups may be bonded to an annular member or to a substituent bonded to an annular member, e.g., carboxyalkyl, methoxyethyl, methoxymethyl, aminoethyl, dialkylaminopropyl, polyoxyethylene, polyaminoethylene, etc. In many cases, for annular nitrogen substituents, conveniently, they will be substituted with an alkyl group of from 1 to 3 carbon atoms, particularly methyl, and at least one adjacent annular carbon atom unsubstituted.

For the most part, individual substituents will be under 600 Da, usually under about 300 Da, and preferably under about 150 Da, and the total for substituents bonded to annular members will be under about 5 kDa, usually under about 2 kDa, more usually under about 1 kDa, there generally being from about 0 to 5, more usually from about 0 to 3 substituents, for other than the alkyl of from 1 to 3 carbon atoms bonded to annular nitrogen.

Generally, the total of carbon atoms for the substituents will not be greater than about 100, usually not greater than about 60, more usually not greater than about 30, with not more than 30 heteroatoms, usually not more than 20 heteroatoms, more usually not more than about 10 heteroatoms.

Aliphatic Amino Acids.

In addition to the organic cyclic compounds, aliphatic amino acids are employed, particularly 107 -amino aliphatic amino acids, to provide for hairpin turns to provide complementation between two sequences of heterocycles; to form a cyclic compound where the oligomers are joined at both ends; or to provide for a shift in spacing of the organic cyclic compounds in relation to the target dsDNA.

For the most part, the aliphatic amino acids will have a chain of two to six carbon atoms, usually of two to four carbon atoms, as a core structure, desirably having terminal amino groups, and being unsubstituted or substituted on carbon and nitrogen, particularly carbon, although for the most part the aliphatic amino acids will be unsubstituted. The same types of substituents that have been described for the heterocycles may also be employed here. Examples of suitable aliphatic amino acids include glycine (“Gly”), β-alanine (“βAla”), and γ-aminobutyric acid (“Gaba”).

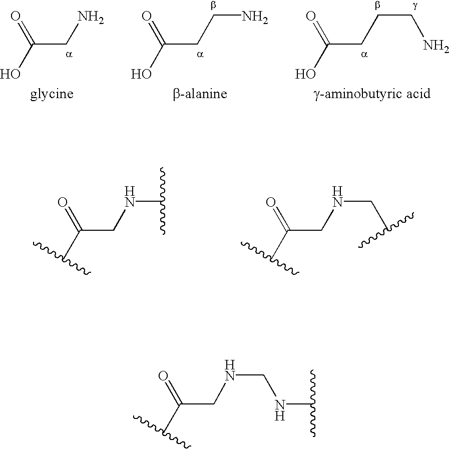

Where an aliphatic amino acid is C-terminal, the carboxyl group (—COOH) will usually be functionalized as an ester (—COOR) or amide (—CONR₂), where the alcohol (ROH) or amine/amino acid (RNH₂) may be selected to provide for specific properties or be used to reduce the charge of the carboxyl group. For the latter, the alcohol and amino groups (R) will generally be from 1 to 6 carbon atoms, usually from 1 to 3 carbon atoms; and from 1 to 6 carbon atoms, usually from 1 to 3 carbon atoms, respectively.

As indicated above, these aliphatic amino acids will play specific roles. The longer chain of aliphatic amino acid will serve to provide for turns in the molecule and to close the molecule to form a ring. The shorter chain aliphatic amino acids will be employed, both to provide a shift for spacing in relation to the target dsDNA, and to provide enhanced binding by being present proximal to the terminal organic cyclic group. The aliphatic amino acid may be present at one or both ends of the oligomer. Of particular interest are glycine and alanine, for space-shifting, β-alanine is preferred. Usually, a consecutive sequence of 6 heterocycles will be avoided. Generally, there will be an amino acid, for example, β-alanine, introduced in an otherwise consecutive series of six oligomer units, generally bordered by at least one, preferably at least two organic cyclic groups, particularly heterocycles.

The aliphatic chains of the aliphatic amino acids may serve as sites of substitution, the aliphatic amino acid providing a core structure, there usually being not more than 2, more usually not more than 1, substituent. The same types of substituents that have been described for the heterocycles may also be employed here.

Conveniently, the substituted aliphatic amino acid may be used in the synthesis of the oligomer, rather than modifying the aliphatic amino acid after the oligomer is formed. Alternatively, a functional group may be present on the chain of the substituent, if necessary being appropriately protected during the course of the synthesis, which functional group may then be used for the subsequent modification. Desirably such functional group could be selectively used, for synthesis of different oligomers, so as to provide for substitution at that site to produce products having unique properties associated with a particular application. With the substituent substituted at a site which does not significantly interfere with the binding in the groove, e.g., employing a single stereoisomer, properties can be imparted to the subject compounds, such as water solubility, lipophilicity, non-covalent binding to a receptor, radioactivity, fluorescence, etc.

Linking Groups.

The heterocycles will normally be linked at the 2 position and the 4 or 5 position, particularly the 2 and 4 position for five annular member rings.

The linking groups between the organic cyclic groups and aliphatic amino acid groups, if present, will generally have a length of two atoms, wherein at least some of the linking groups will have NH, where the NH may hydrogen bond with an unshared pair of electrons of the nucleotides. The linking chains may be methyleneamino (—CH₂—NH—), carboxamide (—C(═O)NH—), ethylene (—CH₂CH₂—), thiocarboxamide (—C(═S)NH—), carboxamidinoyl (—C(═NH)NH—), and the like, particularly carboxamide and its heteroanalogs, e.g., thiocarboxamide and carboxamidinoyl.

A portion of a sequence specific polyamide is illustrated below, showing several units (organic cyclic groups and aliphatic amino acids) linked by carboxamide (—C(═O)NH—) linking groups (in boxes).

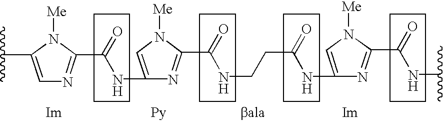

Polar Group.

Optionally, one or both termini of the sequence specific polyamide, preferably one of the termini, will have a polar; group substituted on an alkyl group, where the polar group will generally be from 2 to 6, more usually 2 to 4, carbon atoms from the linkage to the remaining molecule. The polar group may be charged or uncharged, where the charge may be a result of protonation under the conditions of use. Particularly, groups capable of hydrogen bonding are preferred, such as amino, particularly tertiary-amino, hydroxyl, mercapto, and the like. Of particular interest is amino, more particularly alkylated amino, where the alkyl groups are of from 1 to 6, usually 1 to 3, more usually 1, carbon atom, and at a pH less than about 8, the amino group is positively charged and can hydrogen bond the dsDNA.

An example illustrating a terminus of a sequence specific polyamide which includes a terminal polar group, which is a N-substituted,N-methylaminopropyl group, is shown below.

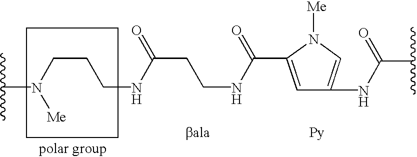

Sequence Specific Polyamide.

In a preferred format, the sequence specific polyamide comprises N-methyl pyrrole (“Py”) and N-methyl imidazole (“Im”) units, using carboxamido groups (—C(═O)NH—) as the linking chains, with the aliphatic amino acids glycine (Gly), β-alanine (βAla) and γ-aminobutyric acid (Gaba), as well as N-substituted aminopropyl (e.g., dimethylaminopropyl, N-substituted,N-methyl-aminopropyl) as the polar substituted alkyl group. These compounds are illustrated as exemplary of the sequence specific polyamides which may be employed in the subject invention.

It is understood that one or a few of the heterocycles may be replaced with a different organic cyclic group, as well one or the other of the aliphatic amino acids may be replaced with a different amino acid, etc.

Of particular interest are compounds which have at least one organic cyclic group, particularly N-methyl imidazole, which has specificity of one nucleotide, which is present as a complementary pair. Usually, the subject compounds will have at least one of these complementary pairs, frequently at least two of these complementary pairs, and generally fewer than 75% of the complementary pairs will have the organic cyclic group having specificity for a single nucleotide.

In the case of the N-methyl imidazole, there will usually be at least one Im/Py pair, desirably not having more than three, frequently having not more than two, of such pairs consecutively, so that there will frequently be not more than three Im's in a row. There will normally be at least one aliphatic amino acid, frequently two aliphatic amino acids, and frequently not more than eight aliphatic amino acids, usually not more than six aliphatic amino acids, more usually not more than about four aliphatic amino acids. Preferably, there will be an amino acid proximal to at least one terminus of the oligomer. The Im/Py pair provide for greater specificity, and when appropriately placed contribute in at least a similar manner to the Py/Py pair to the binding affinity of the dsDNA. Therefore, by appropriate selection of the target sequence, one may optimize for binding affinity and specificity.

It is found that with β-alanine, β-alanine associates with T-A pairs and will usually form a complementary pair with itself. Thus, β-alanine may be used in juxtaposition to T or A and as a complementary pair with itself with a T-A pair.

The binding affinity Kₐ, as determined using the methods described in WO 98/50582, will be greater than 5×10⁸ M⁻¹, usually greater than 10⁹ M⁻¹, preferably greater than about 10¹⁰ M⁻¹, so as to be able to bind to the target sequence at sub-nanomolar concentrations in the environment in which they are used. The difference in affinity with a single mismatch will be at least 3 fold, usually at least 5 fold, preferably at least 10 fold, and frequently greater than 20 fold, and may be 100 fold or more.

Ligand.

The ligand may be any moiety which is capable of directing the conjugate to a cellular or sub-cellular location. Suitable ligands include general nuclear localisation signals, including the SV40 NLS mentioned herein. Ligands include proteins or polypeptides capable of binding a target receptor, including those based on hormones or other protein signalling proteins which bind to a target on the surface of a cell. Ligands may be comprised of hybrid proteins, for example including a component to direct the conjugate to a particular target cell, together with a component to promote uptake of the conjugate by the cell.

Other ligands include insulin, asialoglycoprotein or synthetic analogues thereof for targeting cells generally, transferrin or malaria circumsporozoite protein to target hepatocytes, RGD analogues to target cell surface integrins, and endosomolitic peptide.

Alternatively, the vector may include mixtures of chimeric molecules. For example, ligands with a variety of properties such as specificity for a particular cell type, the ability to promote escape from endosomes, or the ability to have a strong uptake by the cell nucleus (e.g., an NLS) may be used. Two or more of these types of ligands may be used in a single vector of the invention. The proportions of the different types of ligands may be varied according to the particular needs of those of skill in the art.

Cell targeting proteins which may be used either as such or in conjunction with an NLS include proteins which bind to receptors, such proteins including growth factors (VEGF, FGF, PDGF etc). Antibodies or fragments thereof may also be used, e.g., anti-tumour antibodies such as anti-CEA.

The ligand need not be a protein or polypeptide, and may be other chemicals including carbohydrates, such as mannose, used by Ferkol et al. (1996) to target macrophages.

The ligand may be linked to the polyamide through a chemoselective reaction, akin to that described in Muir et al., 1994.

Compositions and Uses of the Invention.

Vectors of the invention may be formulated into compositions wherein the vector is mixed with a pharmaceutically acceptable diluent or carrier. Such compositions form a further aspect of the invention. Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with sterile liquid carriers.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents and the like, such as for example, sodium acetate, sorbitan monolaurate, triethanolamine oleate, triethanolamine sodium acetate, etc.

Such carriers also include lipids, which may be formulated to provide liposome compositions which for delivery of vectors of the invention to target cells.

Vectors and compositions of the invention may be used as medicaments for the treatment of the human or animal body. The vectors and compositions may be delivered to primary cells or cell lines of all types, such as fibroblasts, muscle cells, cells of the nervous system (e.g., neurons, astrocytes, glial cells), hepatocytes, haemopoetic cells (e.g., B- or T-lymphocytes, dendrocytes), epithelial cells, and pluripotent precursor cells such as stem cells, including embryonic stem cells.

The vectors and compositions of the invention may be used to introduce the DNA of the vector into cells in vivo, ex vivo, or in vitro. Where the vector or composition is for in vivo use, it may be delivered to the subject by any suitable means of delivery, such as oral, rectal, nasal, topical (including buccal and sublingual), vaginal or parenteral (including subcutaneous, intramuscular, intravenous, intradermal, intrathecal and epidural) administration. Usually, parenteral administration is preferred, particularly injection to the site of a target tissue, such as a target organ.

Alternatively, ex vivo introduction of the vectors may be used, either on autologous or heterologous cells, and the cells implanted into the patient at the site of desired treatment.

The dose, route and frequency of administration of vectors or compositions of the invention will depend upon a number of parameters including the nature of the condition being treated, the age and condition of the patient, and the like, and will ultimately be at the discretion of the physician.

Thus, another aspect of the present invention pertains to vectors (and compositions comprising vectors), as described herein, for use in a method of treatment of the human or animal body.

Another aspect of the present invention pertains to use of vectors (and compositions comprising vectors), as described herein, for the manufacture of a medicament for the treatment of condition treatable by gene therapy.

Another aspect of the present invention pertains to a method of gene therapy comprising administering to a patient a therapeutically effective amount of a vector (or a composition comprising a vector), as described herein.

Further Aspects of the Invention.

The present invention further provides a method of introducing a dsDNA into a cell or a sub-cellular compartment, said method comprising the steps of:

(a) providing a chimeric molecule comprising:

(i) a sequence specific polyamide moiety capable of binding non-covalently to a target nucleic acid sequence; and,

(ii) a ligand moiety linked covalently to said sequence specific polyamide moiety, and capable of being directed to said cell or said sub-cellular compartment;

(b) providing a dsDNA, said dsDNA including a target sequence for said sequence specific polyamide moiety, under conditions wherein said chimeric molecule binds to said dsDNA to provide a vector; and,

(c) bringing said vector into contact with said cell under conditions for uptake of said vector and transport of said dsDNA.

The present invention further provides a method of introducing a dsDNA into the nucleus of a eukaryotic cell, said method comprising the steps of:

(a) providing a chimeric molecule comprising:

(i) a sequence specific polyamide moiety capable of binding non-covalently to a target nucleic acid sequence; and,

(ii) a ligand moiety linked covalently to said sequence specific polyamide moiety, and capable of being directed to the nucleus of said eukaryotic cell;

(b) providing a dsDNA, said dsDNA including a target sequence for said sequence specific polyamide moiety, under conditions wherein said chimeric molecule binds to said dsDNA to provide a vector; and,

(c) bringing said vector into contact with said eukaryotic cell under conditions for uptake of said vector and transport of said dsDNA.

The invention also provides cells which have been obtained by such a method, and their progeny.

The method of the invention may also be used to provide cell lines which express a protein of interest, for example for the preparation of cells which express high levels of the protein in vitro, to provide for the production of pharmaceutically useful products. Such cells include those used in industry for such production, for example CHO cells.

Synthesis.

The synthesis of the polyamide, the nature of the linker between the domains, and the ligation step has been optimized, with notable and substantial variations with respect to the published protocols. Described herein is a rapid, high-yield procedure for the synthesis of the chimeras, which is amenable to scaling-up. The salient features of the improved methodology, which forms a further aspect of the present invention includes the following.

The use of a “safety-catch linker,” instead of the base-labile linker, in cleaving the polyamide from a solid resin support, allows for a better yield of the resin cleavage step, and increases the range of chemical manipulations at the carboxy terminus of the molecule. Typically, this provides for a reaction of the following type:

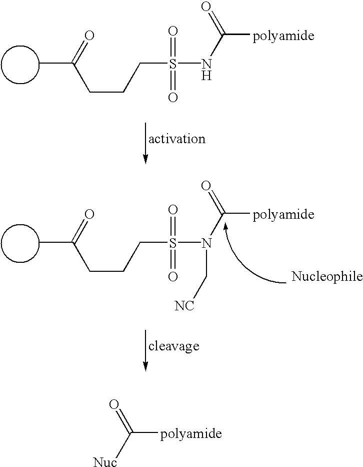

Thus the present invention provides a method for the synthesis of a sequence specific polyamide wherein said polyamide is synthesised on a solid support, said method comprising the steps of:

(a) attaching an N-terminal of the polyamide to the solid support via a safety-catch linker as described herein (e.g., —S(═O)₂—NH—); and,

(b) following synthesis of said polyamide, removing it from said solid support by cleavage of the safety-catch linker by activation and nucleophilic attack.

In one embodiment, the safety-catch linker comprises a linkage —S(═O)₂—NH—. In one embodiment, the safety-catch linker comprises a linkage —CH₂—S(═O)₂—NH—. In one embodiment, the safety-catch linker comprises a linkage —CH₂CH₂—S(═O)₂—NH—. In one embodiment, the safety-catch linker comprises a linkage —CH₂CH₂CH₂—S(═O)₂—NH—. In one embodiment, the safety-catch linker comprises a linkage —C(═O)—CH₂CH₂CH₂—S(═O)₂—NH—.

In one embodiment, the safety-catch linker comprises a linkage —S(═O)₂—NH—C(═O)—. In one embodiment, the safety-catch linker comprises a linkage —CH₂—S(═O)₂—NH—C(═O)—. In one embodiment, the safety-catch linker comprises a linkage —CH₂CH₂—S(═O)₂—NH—C(═O)—. In one embodiment, the safety-catch linker comprises a linkage —CH₂CH₂CH₂—S(═O)₂—NH—C(═O)—. In one embodiment, the safety-catch linker comprises a linkage —C(═O)—CH₂CH₂CH₂—S (═O)₂—NH—C(═O)—.

The safety-catch linker may be introduced using, e.g., a 4-sulfonamidobutyryl resin (Novabiochem). Prior to cleavage, the safety-catch linker is activated: the amine functionality may be substituted, e.g., by a nitrile group (to provide a linker —S(═O)₂—N(CH₂CN)—) or the like.

This may be achieved, for example, by reaction with iodoacetonitrile (I—CH₂—CN). Cleavage may then be achieved by reaction with a nucleophile, which may be base. Examples of suitable nucleophiles include an amine and a thiol.

The safety-catch linker is described in: Backes, B. J.; Virgilio, A. A.; Ellman, J. A. J. Am. Chem. Soc., (1996), Vol. 118, pp. 3055-3056, and Backes, B. J.; Ellman, J. A. J. Org. Chem., (1999), Vol. 64, pp. 2322-2330.

The use of this linker allows for much more flexibility in the choice of the chemistry used. Particular advantages of the safety-catch linker include:

(a) The linker-polyamide bond is stable to both basic and acid conditions, and therefore compatible with both protection schemes (Boc or Fmoc) used for the synthesis of the polyamides. Boc protection is described in WO 98/50582 and other related applications by Dervan et al; Fmoc protection is described in Vásquez, E.; Caamaño, A. M., Castedo, L.; Mascareñas, J. L. Tetrahedron Lett., (1999), Vol. 40, pp. 3621-3624. A combination of Fmoc and Boc protections is also possible, which adds further to flexibility in the design of the synthetic strategy;

(b) More importantly, cleavage of the completed polyamide from the resin by nucleophilic displacement can now be accomplished with stoichiometric amounts of the displacing nucleophile, instead of the large excesses which are necessary when using the standard base-labile linker. The economic use of the displacing nucleophile makes practical the use of more elaborate constructs, e.g., of nucleophiles which incorporate additional features (see compound (15) in the examples) which are important for the subsequent conjugation step; and

(c) Finally, nucleophiles other than bases can be used, e.g., thiols, to produce polyamide C^{α}-thioesters. These are pre-activated for condensation with a peptide containing a suitable residue at the N-terminus, in a reaction called “Native Chemical Ligation,” as described in Dawson, P. E., et al., Science, (1994), Vol. 266, pp. 776-779 and Tam, J. P., Lu, Y. A., and Shao, J., Proc. Natl. Acad. Sci. U.S.A., (1995), Vol. 92, pp. 12485-12489.

Where a thiol is used as a cleavage agent to provide a polyamide, linkage to the polypeptide is via the polypeptide's N-terminal residue, which conveniently may be cysteine. See, for example:

Wilken, J.; Kent, S. B. H. Curr. Opin. Biotechnol., (1998), Vol. 9, pp. 412-426.

Hackeng, et al., Proc. Natl. Acad. Sci. U.S.A., (1997), Vol. 94, pp. 7845-7850.

Lu, W.; Starovasnik, M. A.; Kent, S. B. H. FEBS Lett., (1998), Vol. 429, pp. 31-35.

Wilken, J.; et al. Chem. Biol. (1999), Vol. 6, pp. 43-51.

Kochendoerfer, et al. Biochemistry, (1999), Vol. 38, pp. 11905-11913.

Sydor, J. R.; et al. Proc. Natl. Acad. Sci. U.S.A., (1999), Vol. 96, 7865-7870.

Lee, D. H.; et al. Nature, (1996), Vol.382, pp. 525-528.

Severin, K.; et al. Nature, (1997), Vol. 389, pp. 706-709.

Lee, D. H.; et al. Nature, (1997), Vol. 390, pp. 591-594.

Yao, S.; et al. J. Am. Chem. Soc., (1997), Vol. 119, pp. 10559-10560.

Yao, S.; et al. Angew. Chem. Int. Edn. Engl., (1998), Vol. 37, pp. 478-481.

Yao, S.; et al. Nature, (1998), Vol. 396, pp. 447-450.

However, the amino acid can also be glycine, histidine or methionine. In Canne, L. E.; et al. J. Am. Chem. Soc., (1996), Vol. 118, pp. 5891-5896, it is shown that ligation is possible for both the X-Gly and the Gly-X junctions. See also:

Zhang, L.; Tam, J. P. Tetrahedron Lett., (1996), Vol. 38, pp. 3-6.

Tam. J. P.; Yu, Q. Biopolymers, (1998), Vol. 46, pp. 319-327.

### EXAMPLES

The following are examples are provided solely to illustrate the present invention and are not intended to limit the scope of the invention, as described herein. By way of guidance, the examples are arranged as follows:

Examples 1-4 relate to making components of the sequence specific polyamide as follows:

| | |
|---|---|
| Example 1: | Synthesis of imidazole monomers. |
| Example 2: | Synthesis of pyrrole monomers. |
| Example 3: | Synthesis of the HOAt activated pyrrole |
| | monomer. |
| Example 4: | Synthesis of a pyrrole-imidazole dimer. |

Examples 5-8 relate to the preparation of a sequence specific polyamide on a solid support using a safety catch linker.

| | |
|---|---|
| Example 5: | Loading βAla onto a resin via a safety catch |
| | linker. |
| Example 6: | Synthesis of a polyamide on the loaded resin. |
| Example 7: | Synthesis of a cleavage reagent. |
| Example 8: | Cleavage of polyamide by the cleavage |
| | reagent. |

Examples 9-11 relate to the production of a peptide and its coupling to a polyamide.

| | |
|---|---|
| Example 9: | Synthesis of an activated NLS peptide. |
| Example 10: | Coupling of peptide-polyamide conjugate. |
| Example 11: | An alternative peptide-polyamide coupling |
| | reaction. |

Examples 12 and 13 relate to the preparation of labelled polyamide and peptide.

| | |
|---|---|
| Example 12: | Production of rhodamine and fluorescein |
| | labelled polyamides. |
| Example 13: | Production of rhodamine labelled peptide. |

Example 14 relate to the preparation of a labelled conjugate.

| | |
|---|---|
| Example 14: | Production of labelled conjugate. |

Examples 15-18 relate to the preparation of a mannose cluster and its coupling to a polyamide.

| | |
|---|---|
| Example 15: | Cleavage of polyamide by a cleavage reagent. |
| Example 16: | Derivatisation of the polyamide. |
| Example 17: | Synthesis of a mannose cluster. |
| Example 18: | Conjugation of the mannose cluster to the |
| | derivatised polyamide. |

Examples 19-20 relate to the use of a conjugate of the invention in binding a target DNA sequence.

| | |
|---|---|
| Example 19: | Binding of conjugate to dsDNA |
| | oligonucleotide. |
| Example 20: | Binding of conjugate to plasmid. |

Abbreviations.

The following abbreviations are used herein:

Aoc, aminooctanoic acid;

BOC, tert-butoxycarbonyl;

DCM, dichloromethane;

DIEA, diisopropylethylamine;

DIPC, diisopropylcarbodiimide;

DMAP, N′N′-dimethylamino-pyridine;

DMF, N,N′-dimethylformamide;

DMSO, dimethyl sulfoxide;

Fmoc, 9-fluorenylmethyloxycarbonyl;

Gaba, γ-aminobutyric acid;

HOBt, N-hydroxybenzotriazole;

HOAt, 1-hydroxy-7-azabenzotriazole;

MeOH, methanol;

NMP, N-methylpyrrolidone;

PyBOP, benzotriazol-1-yloxy-tris(pyrrolidino)phosphonium hexafluorophosphate;

TFA, trifluoroacetic acid;

THF, tetrahydrofuran;

TIPS, triisopropylsilane;

WSCD, water-soluble carbodiimide.

General methods.

All the materials were obtained from commercial suppliers and used without further purification. Thin layer chromatography (TLC) was performed in silica gel 60 F₂₅₄ precoated plates (Merck, Darmstadt). Analytical HPLC was performed on a Beckman System Gold chromatograph equipped with a diode-array detector and a Beckmann C-18 column (250×4.6 mm, 5 μm), operating-flow rate 1 mL min⁻¹. Preparative HPLC was performed on a Waters 600E chromatograph equipped with a Jasco UV-975 detector (monitoring wavelength, 254 nm and 214 nm), Waters Delta-Pak™ C-18 column (100×250 mm, 15 μm). The operating flow rate was 30 mL/min. The solvent system was: eluent A, water (0.1% TFA); eluent B, acetonitrile (0.1% TFA). NMR spectra were recorded on a Brucker instrument operating at 400 MHz (¹H). Chemical shifts are reported in ppm relative to the solvent residual signal.

### Example 1

### Synthesis of Building Blocks for Pyrrole-Imidazole Hairpin Polyamides: Imidazole Monomers

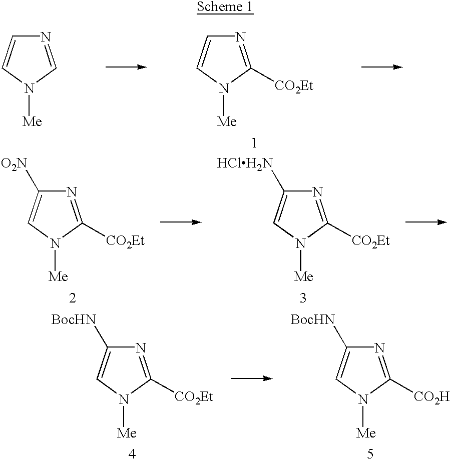

Ethyl 1-methylimidazole-2-carboxylate (1):

N-methylimidazole (50 g, 0.61 mol) was dissolved in 300 mL acetonitrile and 150 mL of triethylamine were added in a 1 L flask equipped with a mechanical stirrer. The solution was cooled to B20 □C, and ethyl chloroformate (106 g, 137.5 mL, 0.97 mol) was added with stirring. The reaction mixture was allowed to slowly warm to room temperature and stirred for 36 h. The precipitated triethylamine hydrochloride was removed by filtration and the solution was concentrated in vacuo. The resulting oil was distilled under reduced pressure (2 torr, 270 Pa, 102-105° C.) to provide the product as a white solid (58 g, 62%). ¹H-NMR (DMSO-d₆) δ: 7.45(s, 1H), 7.05(s, 1H), 4.30(q, 2H), 3.90(s, 3H), 1.30(t, 3H). Ion spray mass spectrometry: calculated for C₇H₁₀N₂O₂ 154 Da, found 155 Da.

Ethyl 1-methyl-4-nitroimidazole-2-carboxylate (2):

Compound (1) (36.0 g, 0.26 mol) was carefully dissolved in 100 mL conc. H₂SO₄ and cooled to 0° C. 90% nitric acid (100 mL) was slowly added, with the temperature maintained at 0° C. The reaction mixture was then refluxed with a dry ice condenser for 50 min and then cooled with an ice bath and quenched by pouring onto 1 L of ice. The resulting blue solution was extracted with 500 mL carbon tetrachloride and then with DCM (3×500 mL). The combined DCM extracts were washed with brine, dried over sodium sulfate and concentrated in vacuo to yield an oil. Crystallisation from 210 mL carbon tetrachloride and 10 mL ethanol provided white crystals (15.0 g, 32%). ¹H-NMR (DMSO-d₆) δ: 8.65(s, 1H), 4.35(q, 2H), 4.00(s, 3H), 1.35(t, 3H). Ion spray mass spectrometry: calculated for C₇H₉N₃O₄ 199 Da, found 200 Da.

Ethyl 4-amino-1-methylimidazole-2-carboxylate hydrochloride (3):

The nitroimidazole ethyl ester (2) (10.3 g, 52 mmol) was dissolved in 500 mL of ethanol/ethyl acetate (1:1 v/v). 10% Pd/C (2.00 g) was added as a slurry in 50 mL of ethyl acetate and the mixture was stirred under a slightly positive hydrogen pressure. After 12 hr, TLC (silica, petroleum ether/ethyl acetate 7:3, UV 254 nm) showed complete disappearance of the starting material. The reaction mixture was filtered and concentrated in vacuo to 50 mL and then 500 mL diethyl ether were added. Addition of HCl gas provided a white precipitate. The solution was cooled to B20° C. for 4 hr and the precipitate filtered off. Drying in vacuo afforded 7.40 g (69%) of a yellow solid. ¹H-NMR (DMSO-d₆) δ: 10.00(br s, 1H), 7.35(s, 1H), 4.25(q, 2H), 3.90(s, 3H), 1.25(t, 3H). Ion spray mass spectrometry: calculated for C₇H₁₁N₃O₂ 169 Da, found 170 Da.

Ethyl 4-[(tert-butoxycarbonyl)amino]-1-methylimidazole-2-carboxylate (4):

The imidazole amine (3) (7.26 g, 35.4 mmol) was dissolved in 20 mL DMF. DIEA (4.5 mL, 49.1 mmol) was added followed by di-tert-butyl dicarbonate (9.60 g, 47.6 mmol). The mixture was stirred at 60° C. for 18 hr, allowed to reach room temperature and partitioned between 50 mL brine and 50 mL diethyl ether. The ether layer was extracted with 10% citric acid (2×20 mL), brine, saturated sodium bicarbonate, and brine, then dried over sodium sulfate and concentrated in vacuo to yield 8.11 g of Boc ester, contaminated with Boc anhydride. ¹H-NMR (DMSO-d₆) δ: 9.70(s, 1H), 7.30 (s, 1H), 4.25(q, 2H), 3.90(s, 3H), 1.45(s, 9H), 1.30(t, 3H). Ion spray mass spectrometry: calculated for C₁₂H₁₉N₃O₄ 269 Da, found 270 Da.

4-[(tert-butoxycarbonyl)amino]-1-methylimidazole-2-carboxylic acid (5):

The ester (4) (4.00 g, 14.8 mmol) was dissolved in 80 mL water/methanol (1:1 v/v) 1 M NaoH and stirred at 40° C. for 4 hr. TLC (silica, DCM/methanol 9:1) showed complete disappearance of the starting material. The reaction mixture was cooled to 0° C. and the pH value carefully adjusted to 3 with 10% NaHSO₄. The aqueous layer was extracted with ethyl acetate (10×150 mL). The collected organic layers were washed with brine, dried over sodium sulfate and concentrated in vacuo to yield 2.56 g (72%) of white solid. ¹H-NMR (DMSO-d₆) δ: 9.60(s, 1H), 7.25 (s, 1H), 3.85(s, 3H), 1.40(s, 9H). Ion spray mass spectrometry: calculated for C₁₀H₁₅N₃O₄ 241 Da, found 242 Da.

### Example 2

### Synthesis of Building Blocks for Pyrrole-Imidazole Hairpin Polyamides: Pyrrole Monomers

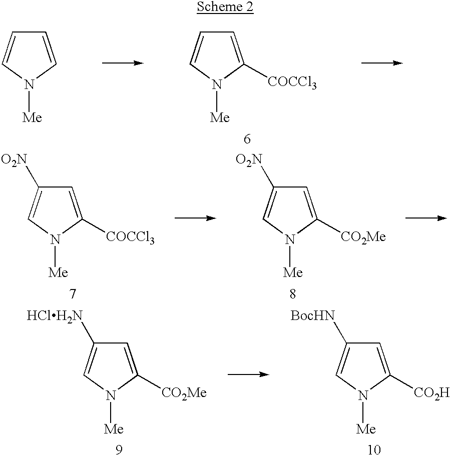

2-(trichloroacetyl)-1-methylpyrrole (6):

To a well-stirred solution of trichloroacetyl chloride (100 g, 0.55 mol) in 150 mL diethyl ether, a solution of N-methylpyrrole (45 gr, 0.54 mol) in ether (150 mL) was added dropwise. The mixture was stirred for an additional 3 hr and the reaction was quenched by dropwise addition of a solution of 40 g potassium carbonate in 150 mL water. The layers were separated and the ethereal one was concentrated in vacuo. Trituration with petroleum ether yielded 10.5 g (85%) of (6) as a yellow solid. ¹H-NMR (DMSO-d₆) δ: 8.50(s, 1H), 7.75 (s, 1H), 3.95(s, 3H).

4-nitro 2-(trichloroacetyl)-1-methylpyrrole (7):

To a cooled (−40° C.) solution of (6) (2.40 g, 10 mmol) in acetic anhydride (12 mL), 0.8 mL of fuming nitric acid was added dropwise. The reaction mixture was allowed to reach room temperature and stirred for an additional 4 hr. The solvents were distilled off in vacuo, and the residue solidified on standing. Trituration with diethyl ether afforded 1.97 g (73%) of (7) as a yellow solid. ¹H-NMR (DMSO d₆) δ: 7.45(d, 1H), 7.45 (d, 1H), 6.3(dd, 1H), 3.90(s, 3H).

Methyl 4-nitropyrrole-2-carboxylate (8):

To a solution of (7) (8.00 g, 29.5 mmol) in 25 mL dry methanol a solution of 30% MeONa in MeOH (0.5 mL, 2.5 mmol) was added dropwise. The resulting mixture was stirred at room temperature for 2 hr and then quenched by addition of 0.25 mL of concentrated sulfuric acid. The solution was then heated to reflux and allowed to slowly cool to room temperature. White crystals formed, which were filtered and dried in vacuo to afford 5.4 g (100%) of (8). ¹H-NMR (DMSO-d₆) δ: 8.25(s, 1H), 7.25 (s, 1H), 3.90(s, 3H), 3.75(s, 3H). Ion spray mass spectrometry: calculated for C₇H₈N₂O₄ 184 Da, found 185 Da.

Methyl 4-amino-1-methylpyrrole-2-carboxylate hydrochloride (9):

Nitropyrrole (8) (4.00 g, 22 mmol) was dissolved in ethyl acetate (100 mL). 1.0 g of 10% Pd/C was added as a slurry in 10 mL ethyl acetate and the mixture was stirred under a slightly positive hydrogen pressure. TLC (silica, DCM/methanol 9:1) after 24 hr showed complete disappearance of the starting material. Pd/C was removed by filtration through celite and the volume reduced to 20 mL by concentration in vacuo. Diethyl ether was added (70 mL) and HCl gas was gently bubbled through the mixture. The precipitated amine hydrochloride was collected by vacuum filtration to yield 3.86 g (92%) of (9) as a beige powder. ¹H-NMR (DMSO-d₆) δ: 10.00(s_{br}, 3H), 7.25 (s, 1H), 6.80(s, 1H), 3.85(s, 3H), 3.70(s, 3H). Ion spray mass spectrometry: calculated for C₇H₁₀N₂O₂ 154 Da, found 155 Da.

4-[(tert-butoxycarbonyl)amino]-1-methylpyrrole-2-carboxylic acid (10):

The hydrochloride (9) (11.58 g, 60.7 mmol) was dissolved in 145 mL 10% aqueous sodium carbonate; di-tertbutyl carbonate (19.3 g, 103 mmol) was added dropwise as a slurry in 36 mL dioxane. The reaction was stirred at room temperature for 2 hr, then cooled to 4° C. The resulting white precipitate was collected by vacuum filtration. The crude material was dissolved in 150 mL 1M NaOH/methanol (1:1 v/v) and the solution was heated at 60° C. for 6 hr. The solution was cooled at room temperature and washed with diethyl ether (2×250 mL). The pH of the aqueous layer was reduced to 3 with 10% NaHSO₄ and the mixture extracted with ethyl acetate (4×250 mL). The combined ethyl acetate extracts were dried over sodium sulfate and concentrated in vacuo to provide a yellow foam (8.73 g, 60%). ¹H-NMR (DMSO-d₆) δ: 12.00(s, 1H), 9.00 (s, 1H), 7.00(s, 1H), 6.55(s, 1H), 3.75(s, 3H), 1.40(s, 9H). Ion spray mass spectrometry: calculated for C₁₁H₁₆N₂O₄ 240 Da, found 241 Da.

### Example 3

### Synthesis of Building Blocks for Pyrrole-Imidazole Hairpin Polyamides: Activated Pyrrole Monomer

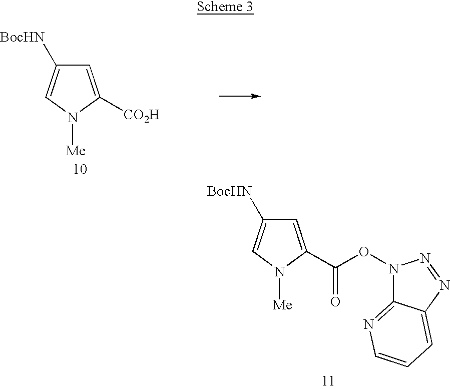

4-[(tert-butoxycarbonyl)amino]-1-methylpyrrole-2-carboxylic HOAt ester (11):

A suspension of pyrrole (10) (1.00 g, 4.16 mmol), HOAt (679 mg, 1.2 eq) and DMAP (51 mg, 0.1 eq) in 15 mL DCM was cooled in an ice-bath under Ar. A suspension of WSCD (957 mg, 1.2 eq) in 15 mL DMC was added dropwise. After 5 min everything solubilized, and stirring was continued for 12 hr at room temperature. A white solid formed which was filtered off to yield 1.46 g (98%) of pure product. ¹H-NMR (DMSO-d₆) δ: 9.40(s, 1H), 8.80(d, 1H), 8.70(d, 1H), 7.65(dd, 1H), 7.55(s, 1H), 7.20(s, 1H), 3.80(s, 3H), 1.45(s, 9H). Ion spray mass spectrometry: calculated 358 Da, found 359 Da.

### Example 4

### Synthesis of Building Blocks for Pyrrole-Imidazole Hairpin Polyamides: Pyrrole-Imidazole Dimer

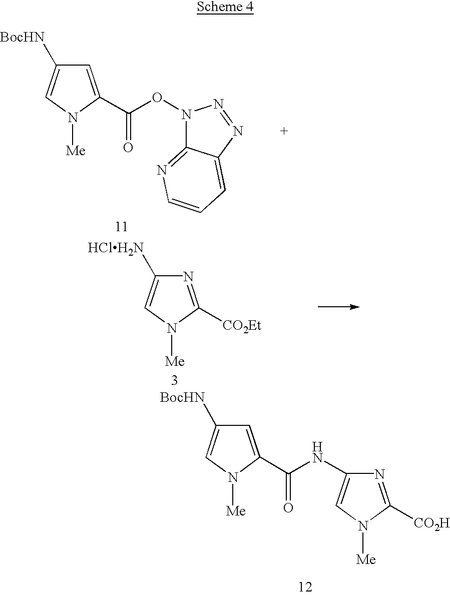

BOC-Pyr-Im-OH (12):

A solution of (11) (559 mg, 1.56 mmol), (3) (321 mg, 1.56 mmol), and DIEA (0.27 mL, 1.56 mmol) in 5 mL DMF was stirred and heated at 60° C. for 6 hr. Dilution with ice-water induced formation of a white precipitate, which was filtered off. The solid was dissolved in 20 mL of (1:1 v/v) MeOH/1 M NaOH and heated at 60° C. for 12 hr. The solution was cooled to room temperature and carefully acidified with 1 N HCl: a white precipitate formed and was filtered off. After drying in vacuo 620 mg of (12) (86%) were obtained as a white solid. ¹H-NMR (DMSO-d₆) δ: 10.55(s, 1H), 9.05(s, 1H), 7.60(s, 1H), 7.00(s, 1H), 6.90(s, 1H), 3.90(s, 3H), 3.80(s, 1H), 1.15(s, 9H). Ion spray mass spectrometry: calculated for C₁₆H₂₁N₅O₅ 363 Da, found 364 Da.

### Example 5

### Loading of the First Residue (βAla) onto 4-Sulfonamidobutyryl Resin

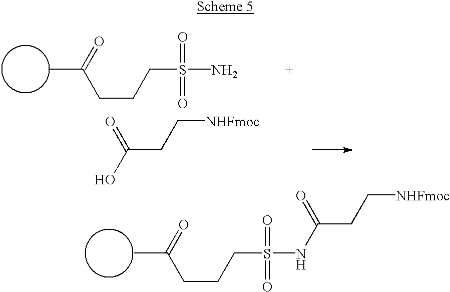

Fmoc-βAla-OH (1.68 gr, 5.4 mmol) and DIPC (0.42 mL, 2.7 mmol) were dissolved in 10 mL DCM and stirred at room temperature for 30 min. The solution was then filtered and added to 1.0 g of 4-sulfonamidobutyryl resin (Novabiochem, substitution 0.9 mmol/g) previously swollen in DCM, together with 330 mg of DMAP (2.7 mmol). The mixture was stirred for 1 hr and then sequentially washed with DCM, DMF, DCM. The procedure was repeated to obtain, after drying in vacuo for 2 hr, 1.36 g of resin.

Evaluation of Loading:

7.5 mg of the resin were treated with 1 mL piperidine and 1 mL DCM for 30 min, then diluted with 19 mL DCM and 4 mL MeOH. The absorbance at 301 nm (corrected for blank absorption) was converted in mmol/g according to the formula A₃₀₁/7800× (25 mL/g of resin): the load of βAla was 0.36 mmol/g.

### Example 6

### Subsequent Solid-Phase Synthesis of Polyamides

The following general synthetic procedures were used, based on a quantity of resin with 0.1 mmol of product loaded.

Removal of the Fmoc Group:

The resin was pre-washed with 2 mL 20% piperidine in DMF and then treated with 5 mL of the same mixture for 20 min, followed by washes with DMF, DCM, 10% TFA in DCM and DCM, until the solution did not test acidic.

BOC Cleavage:

The resin was pre-washed with 1 mL of the cleavage cocktail (80% TFA in DCM, 0.5 M thiophenol), drained and treated with a further 2 mL of cleavage cocktail for 5 min. After draining, another 4 mL of cleavage solution were added and stirring continued for 20 min. The resin was then washed with DCM (100 mL) and DMF (50 mL).

Neutralisation of the Amino Groups:

The resin from BOC cleavage was treated with 400 μL of DIPEA in 2 mL of DMF, stirred for 5 min, drained and immediately used for the next coupling.

Coupling of BOC-Imidazole Units:

96 mg of (5) (0.4 mmol) and 55 mg of HOAt (0.4 mmol) were dissolved in 4 mL DMF together with 77 mg (0.4 mmol) of WSCD and 5 mg (0.04 mmol) of DMAP. The resulting solution was shaken for 10 min, and then added to the resin pre-swollen in DMF. Shaking was continued for 1 hr and then the resin was washed with 50 mL DMF and 50 mL DCM.

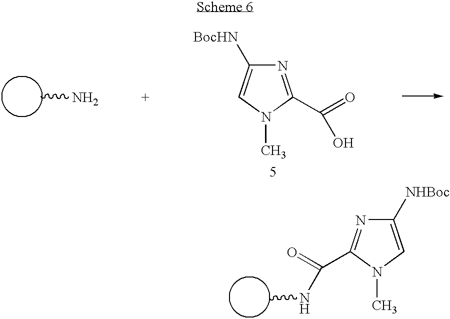

Coupling of HOAt Activated BOC-Pyrrole Units:

143 mg of (11) (0.4 mmol) and 68 μL DIEA (0.4 mmol) were dissolved in 4 mL DMF and added to the resin pre-swollen in DMF. The mixture was shaken for 1 hr and then washed with 50 mL DMF and 50 mL DCM.

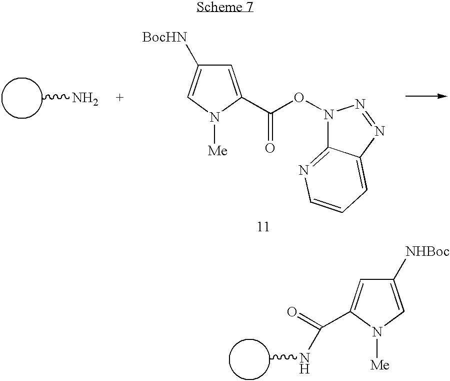

(This method is not preferred for the coupling of a pyrrole on an imidazole free amino group. In such cases, it is preferred to use a pre-formed dimer, for example, (12).)

Coupling of BOC-Pyrrole-Imidazole Units:

363 mg of (12) (0.4 mmol) and 55 mg of HOAt (0.4 mmol) were dissolved in 4 mL DMF together with 77 mg (0.4 mmol) of WSCD and 5 mg (0.04 mmol) of DMAP. The resulting solution was added shaken 10 min, and then added to the resin pre-swollen in DMF. Shaking was continued for 1 hr and then the resin was washed with 50 mL DMF and 50 mL DCM.

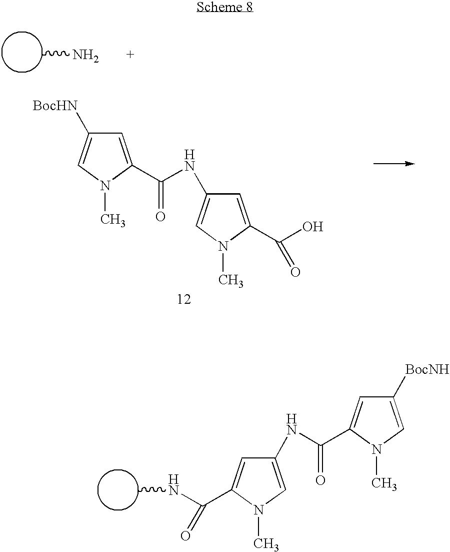

Coupling of BOC-βAla-OH:

76 mg of BOC-βAla-OH (0.4 mmol) and 55 mg of HOAt (0.4 mmol) were dissolved in 4 mL DMF together with 77 mg (0.4 mmol) of WSCD and 5 mg (0.04 mmol) of DMAP. The resulting solution was added to the resin pre-swollen in DMF. The mixture was shaken for 1 hr and then washed with 50 mL DMF and 50 mL DCM.

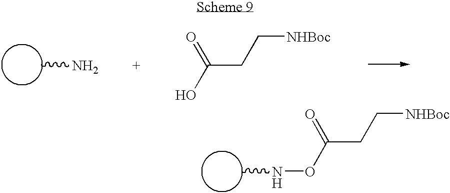

Coupling of BOC-Gaba-OH:

81 mg of BOC-Gaba-OH (0.4 mmol) and 55 mg of HOAt (0.4 mmol) were dissolved in 4 mL DMF together with 77 mg (0.4 mmol) of WSCD and 5 mg (0.04 mmol) of DMAP. The resulting solution was added to the resin pre-swollen in DMF. The mixture was shaken for 1 hr and then washed with 50 mL DMF and 50 mL DCM.

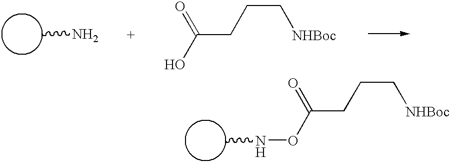

### Example 7

### Preparation of Thiol Amine Derivatives for Cleavage from the Safety-Catch Linker

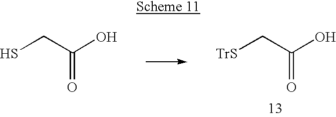

2-(triphenylmethylthio)ethanoic Acid (13):

The compound was prepared as described in Can. J. Chem., Vol. 74, 1503 (1996). Briefly, triphenylcarbinol (12.0 g, 46.1 mmol) and mercaptoethanoic acid (4.25 g, 46.1 mmol) were dissolved in 50 mL 1:1 DCM/acetic acid. BF₃ Et₂O (8 mL, 65 mmol) was added dropwise and stirring was continued for 1 hr. The solvents were removed in vacuo. Addition of 50 mL water induced formation of a white precipitate which was filtered off, washed with water (2×50 mL) and acetonitrile (25 mL) and dried in vacuo to obtain 10.3 g (67%) of product (13). ¹H-NMR (DMSO-d₆) δ: 12.60(s, 1H), 7.30(m, 15H), 2.80(s, 2H). Ion spray mass spectrometry: calculated for C₂₁H₁₈O₂S₃ 334 Da, found (M⁻) 333 Da.

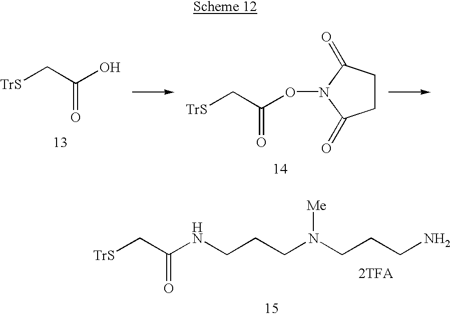

Mono 2-(triphenylmethylthio)ethanoyl Derivative of 3,3′-diamino-N-methyldipropylamine (15):

Acid (13) (1.00 g, 3.00 mmol) and N-hydroxysuccinimide (350 mg, 3.00 mmol) were dissolved in 10 mL THF. A solution of DCC (680 mg, 3.30 mmol) in THF (5 mL) was added dropwise. After 2 hr, TLC analysis (silica, DCM/methanol 9:1) showed complete disappearance of the starting material. The solution was filtered and concentrated in vacuo. The residue was dissolved in 5 mL DCM, filtered and concentrated again to obtain a white solid (14). The solid was dissolved in 20 mL DCM and added dropwise to a stirred solution of 3,3′-diamino-N-methyldipropylamine (6 mL) in 6 mL DCM. TLC analysis (silica, DCM/methanol 9:1) after 1 hr showed complete disappearance of the starting activated ester. The solution was diluted with DCM and washed with 2 N NaOH (twice) and brine, then concentrated. The resulting oily residue was dissolved in 1% TFA/DCM, concentrated and redissolved until the resulting solution remained acidic. Prolonged concentration in vacuo yielded a white foam. The product was purified by RP HPLC, using a linear gradient between 15%-40% of B in 25 min. ¹H-NMR (DMSO-d₆) δ: 8.05 (t, 1H), 7.30 (m, 15H), 3.20-2.80 (m, 8H), 2.80 (s, 2H), 2.70 (s, 3H), 1.90 (m, 2H), 1.70 (m, 2H). Ion spray mass spectrometry: calculated for C₂₈H₃₅N₃O S 461 Da, found 462 Da.

### Example 8

### Cleavage of the Polyamide (16) from the Resin with Thiol Amine Derivative

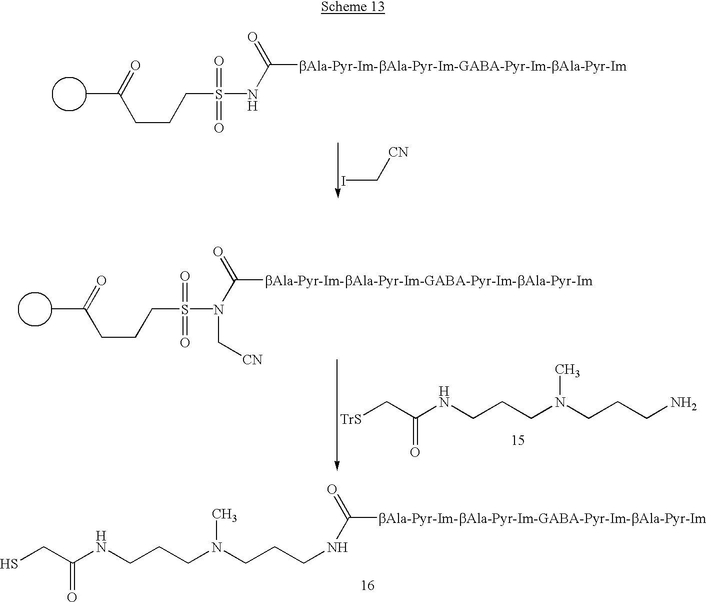

A batch of resin containing the sequence (Resin)-βAla-Pyr-Im-βAla-Pyr-Im-GABA-Pyr-Im-βAla-Pyr-Im, synthesized according to the above procedure (154 mg, 19.2 μm th.) was treated with 0.72 mL (13 mmol) iodoacetonitrile and 0.42 mL (2.4 mmol) DIEA in 2 mL NMP for 4 hr, then washed with NMP, DCM and dried under nitrogen. A solution of (15) (52 mg, 75 μmol) and DIEA (56 μL, 327 μmol) in 0.4 mL DMF was then added and the resin was stirred overnight at room temperature. The solution was filtered and concentrated in vacuo. The oily residue was dissolved in 5 mL DMF and treated with 200 mg PS-isocyanate resin (loading 1.4 mmol/g, 280 μmol) for 2 hr. Filtration and concentration in vacuo afforded a yellow oil. The residue was dissolved in 10 mL of a TFA/DCM (1:1 v/v) solution containing 10% TIPS and stirred for 30 min. Concentration in vacuo yielded an oily residue, which was triturated with diethylether to obtain 95 mg of a yellow oil.

Purification by Size-Exclusion Chromatography:

The yellow oil was re-suspended in 2 mL of DMSO and purified by size-exclusion chromatography onto a 26×800 mm column, slurry packed with TSKgel TOYOPEARL HW-40 (S, 25-40 mm, TosoHaas), using as eluent H₂O/CH₃CN, 60/40, 0.1% TFA at a flow rate of 1 mL/min. The fractions eluted were analyzed by analytical HPLC and the desired product (16) was collected in two pools: >95% pure (16 mg, 28.4%), and 59% pure (9 mg, 16%). The chomatographic yield, calculated as percentage of product recovered versus total product present in the crude material loaded, was 80%. The purified product was analyzed by ion spray mass spectrometry and gave the expected molecular weight: calculated (average isotopic composition) 1483.6 Da, found 1483 Da.

### Example 9

### Synthesis of Bromoacetyl-NLS Peptide NH₂-Pro-Lys-Lys-Lys-Arg-Lys-Val-Glu-Asp-Pro-Tyr-Lys(Br—CH₂—CO)—CO—NH₂ (17)

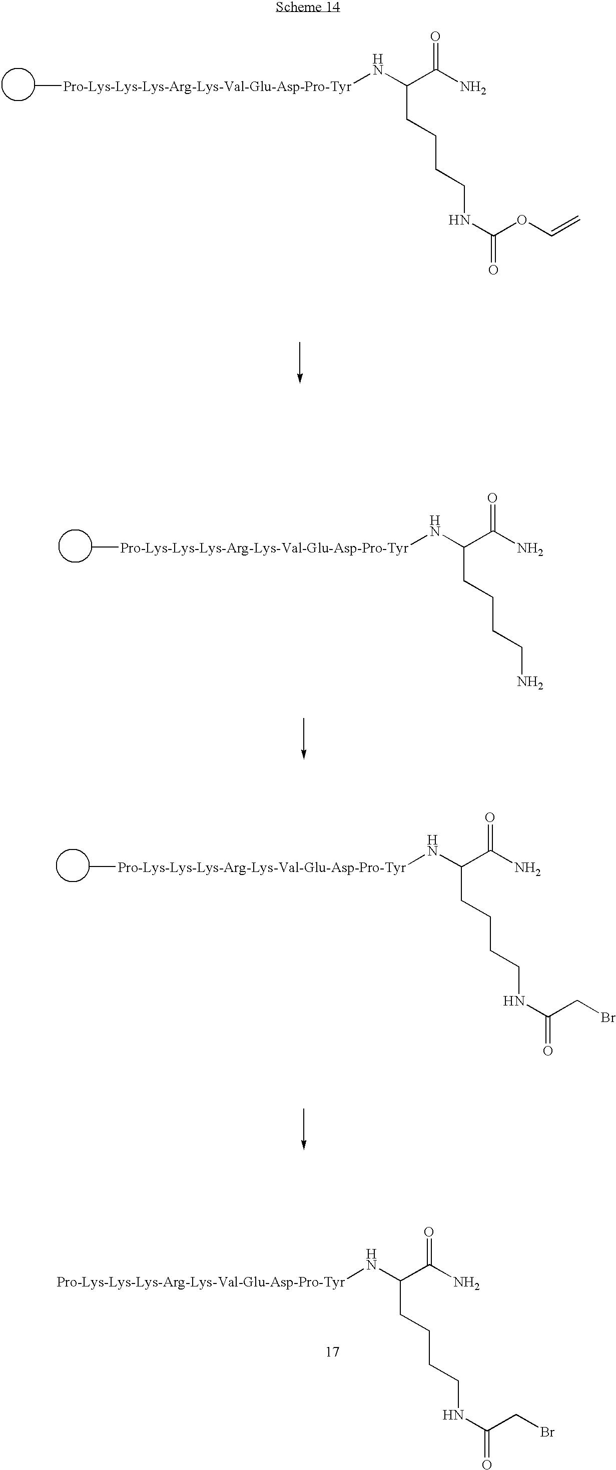

Peptide Assembly:

The peptide H₂N-PKKKRKVEDPY-COOH was synthesized by Fmoc-t-Bu chemistry on a Millipore 9050 Plus synthesizer on 0.5 g of Fmoc-PAL-PEG-PS resin 0.19 meq/g (PE PerSeptive). Side-chain protection was as follows: Fmoc-Arg(2,2,4,6,7-pentamethyldihydrobenzofuran-5-sulfonyl), Fmoc-Asp(O-t-butyl), Fmoc-Glu(O-t-butyl), Fmoc-Lys(t-butoxycarbonyl), Fmoc-Lys(allyloxycarbonyl) for the C-terminal lysine, Fmoc-Tyr(t-butyl). The N-terminal proline was incorporated as the BOC derivative. The protected amino acid (1 eq) was pre-activated with PyBOP (1 eq), HOBt (1 eq), and DIEA (2 eq) using a 5-fold excess of acylant over the resin amino groups. Coupling times were 60 min. At the end of the assembly the resin was washed with DMF, MeOH, diethylether and dried in vacuo.

Cleavage of N^{ε}-allyloxycarbonyl Protecting Group of the C-Terminal Lysine:

The dried peptide resin was treated overnight with 10 mL of a solution of tetrakis(triphenylphosphine) palladium(0), 0.07 M in CHCl₃ containing 5% acetic acid and 2.5% N-methylmorpholine. The resin was then drained and washed with DMF and repetitively with a solution of 0.5% DIEA and 0.5% sodium diethyldithiocarbamate in DMF.

Coupling of Bromoacetic Acid:

The N^{ε} amino group of the C-terminal lysine was reacted with bromoacetic acid (1 eq), DIPC (1 eq), HOBt (1 eq) for 45 min (3-fold excess of acylant). The resin was washed with DMF, MeOH, diethylether and dried in vacuo.

Cleavage of Bromoacetyl NLS Peptide (17) from the Resin:

The peptide resin was treated with 20 mL of TFA 88%, phenol 5%, triisopropylsilane 2%, water 5% (Reagent B) for 2 hr. The resin was filtered and rinsed with TFA. The TFA solution was added dropwise to screw cap centrifuge tubes containing cold methyl t-Bu ether (MTBE) with a TFA/MTBE ratio of 1/10; after centrifugation at 3200× g (30 min), the ether solution was removed and the peptide precipitate re-suspended in 50 mL of MTBE: the process was repeated twice. The dried precipitate was dissolved in CH₃CN/water and lyophilized.

Purification of Bromoacetyl NLS Peptide (17):

The peptide was purified by preparative HPLC on a Waters Delta-Pak C-18 column (20×200 mm). In a typical run, the crude peptide (22 mg) was dissolved in water, 0.1% TFA, loaded onto the preparative column and eluted with a linear gradient between 5%-25% in 20 min at a flow rate of 30 mL/min. The fractions containing the desired peptide (>98% pure) were pooled and lyophilized, yield 6 mg (27%). Ion-spray mass spectrometry of the HPLC purified peptide gave the expected molecular weight: calculated (average isotopic composition) 1636.7 Da, found 1636.4 Da.

### Example 10

### Synthesis of the Peptide/Polyamide Conjugate NLS (18)

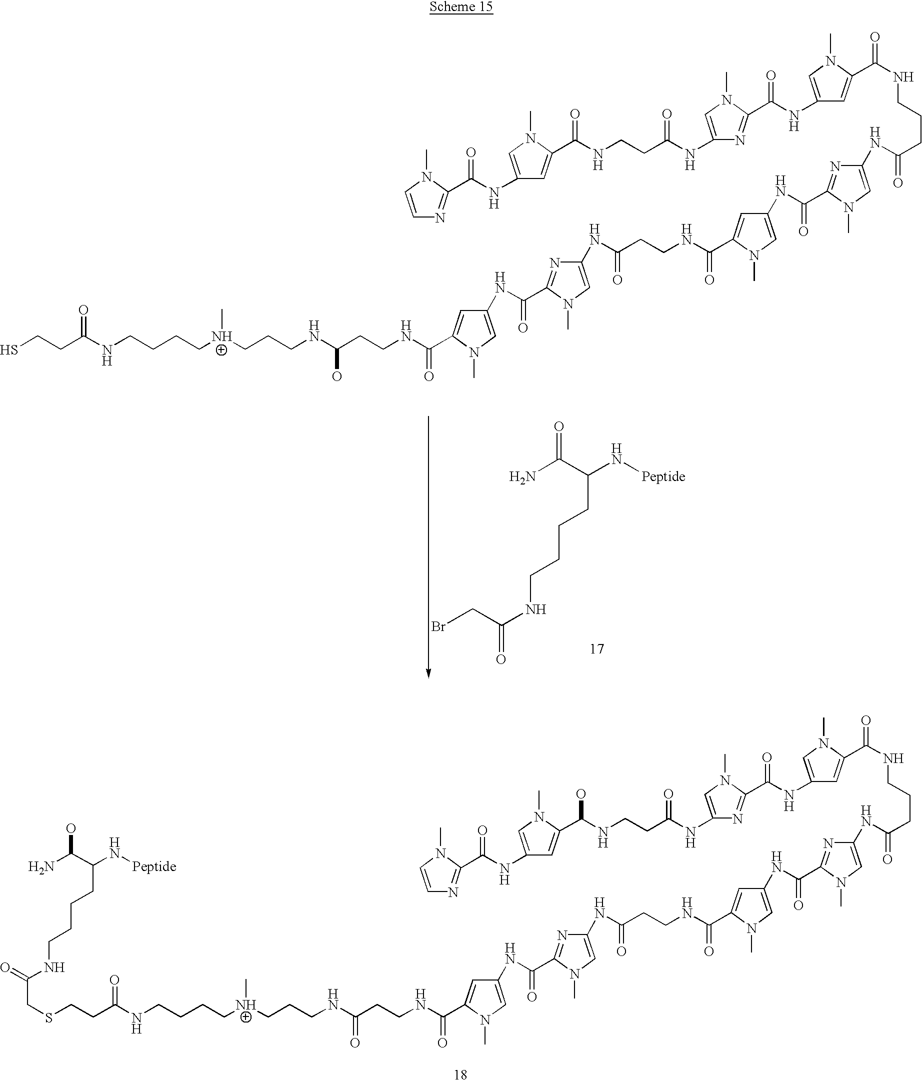

The bromoacetyl-NLS peptide (17) (16 mg), and derivatized polyamide (16) (5 mg), were dissolved in 1 mL of DMF and 10 μL DIEA. The reaction was monitored by analytical HPLC. After 30 mmn the reaction was complete and the solution was immediately purified by preparative HPLC, using a linear gradient between 10%-40% of B in 25 min, with a flow rate of 30 mL/min. The fractions containing the desired product were collected and freeze-dried, yielding 1.5 mg (15%) of (18). Ion-spray mass spectrometry gave the expected molecular weight: calculated (average isotopic composition) 3037.65 Da, found 3037.75 Da.

### Example 11

### Synthesis of Peptide-Polyamide Conjugates by Native Chemical Ligation

Peptide-polyamide conjugates can also be produced by a reaction known as “Native Chemical Ligation”, as described in: (a) Dawson, P. E.; Muir, T. W.; Clark-Lewis, I.; Kent, S. B. H. Science 1994, 266, 776-779. (b) Tam, J. P.; Lu, Y. A.; Shao, J. Proc. Natl. Acad. Sci. U.S.A. 1995,92, 12485-12489. According to this method, one of the components of the ligation reaction (a peptide in the above mentioned papers) is in the form of a C^{α}-terminal thioester, which is pre-activated for condensation with another peptide containing a suitable residue at the N-terminus. Usually the N-terminal residue is a cysteine, but other amino acids can also be used, e.g., Gly (shown in: Canne, L. E.; Bark, S. J.; Kent, S. B. H. J. Am. Chem. Soc. 1996, 118, 5891-5896) His (shown in: Zhang, L.; Tam, J. P. Tetrahedron Lett. 1996, 38, 3-6) and Met (shown in: Tam. J. P.; Yu, Q. Biopolymers 1998, 46, 319-327). The method can be run in aqueous solution at neutral pH and yields a normal peptide (amide) bond at the junction site.

The same reaction can be used to chemoselectively conjugate a polyamide to a peptide, if the polyamide is obtained as C^{α}-terminal thioester and the peptide is synthesized with a N-terminally added suitable amino acid (e.g., cysteine). The latter is prepared by standard solid-phase methods.

The procedure used to prepare the polyamide thioester, which is detailed below, is analogous to the one previously described in Example 8, and makes use of the safety-catch linker using a thiol as nucleophile. The method described for peptides in: Ingenito, R.; Bianchi, E.; Fattori, D.; Pessi, A. J. Am. Chem Soc. 1999, 121, 11369 has thus been extended to minor-groove binding hairpin polyamides.

Cleavage of the Polyamide from the Resin with Thiols to Form C-Terminal Thioester:

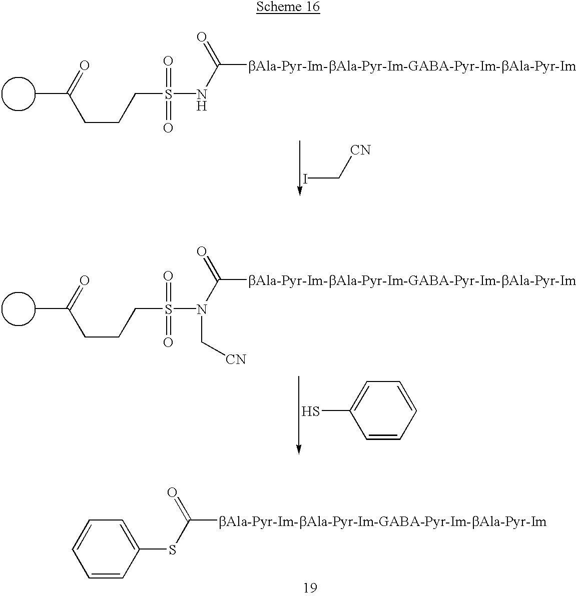

20 mg of resin containing the sequence (Resin)-βAla-Pyr-Im-βAla-Pyr-Im-GABA-Pyr-Im-βAla-Pyr-Im, synthesized according to the above mentioned procedure (6.8 μmol based on initial βAla load) was treated with 0.3 mL (5.4 mmol) iodoacetonitrile and 0.17 mL (0.95 mmol) DIEA in 2 mL NMP for 4 hr, then washed with NMP, DCM and dried under nitrogen. A solution of 0.4 mL thiophenol (3.89 mmol) in 1.0 mL DMF was then added and the resin was stirred for 12 hr at room temperature. The solution was filtered and concentrated in vacuo. Trituration of the solid residue with diethyl ether afforded 10 mg (41.6%) of the crude desired product (19). This was used without further purification in the subsequent reaction. Ion spray mass spectrometry: calculated (average isotopic composition) 1398 Da, found 1399 Da.

Native Chemical Ligation of Polyamide Thioesters:

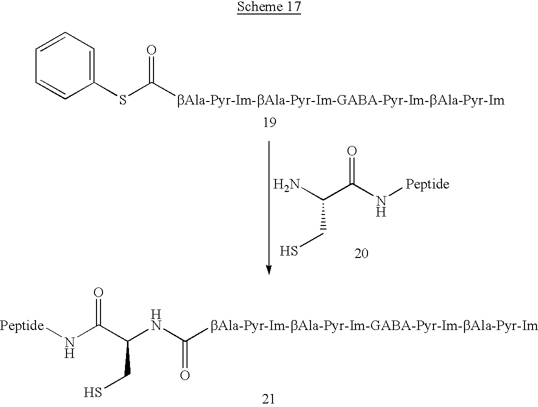

1.3 mg (0.9 μmol) of peptide CMVDYPYRL-NH₂ (Cys-met-Val-Asp-Tyr-Pro-Tyr-Arg-Lys-NH₂) (20) was dissolved in 0.4 mL water, and the pH was adjusted to 6.0 with diluted NaOH. This solution was mixed with a solution of the crude thioester (19) (3.0 mg, 2.14 μmol) in 1 mL DMF, containing 1% thiophenol. The resulting solution was stirred at room temperature, and progress of the reaction was monitored by HPLC (RP C18, Symmetry column 4.6×150 mm). After completion of the reaction, the solution was diluted with 5 mL acetonitrile (containing 0.1% TFA) and concentrated in vacuo to obtain 43 mg of oily residue, which after repeated washes with diethyl ether was reduced to 7 mg. Trituration of the residue with water, followed by centrifugation and lyophilization from (1:1) acetonitrile/water yielded 2.2 mg of crude conjugate (21).

The conjugate was purified by preparative HPLC: column RP-C18, 250×4.6 mm, 5 μm, flow rate 1 mL/min, eluent A, water (0.1% TFA), eluent B acetonitrile (0.095% TFA) sample loaded in neat DMSO at 100% eluent A, then linear gradient from 0% to 60% eluent B in 50 min. The fractions containing the desired compound were pooled and lyophilized, yielding 0.4 mg (0.16 μmol, 18%) of (21). Ion spray mass spectrometry: calculated (average isotopic composition) 2423 Da, found 2423 Da.

### Example 12

### Synthesis of Rhodamine and Fluorescein Labeled Polyamides

Synthesis of Rhodamine Derivative Suitable for Ligation with Polyamide Thiols:

Synthesis of 5(6)-carboxytetraethylrhodamine (24):

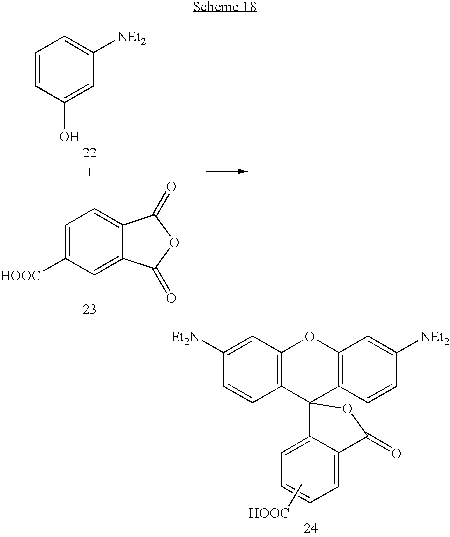

A suspension of tri-mellitic anhydride (23) (4.8 g, 24 mmol) in xylene (100 mL) was heated at reflux while stirring. A solution of 3-diethylaminophenol (22) (8.2 g, 49 mmol) in xylene (50 mL), was added dropwise. The mixture was kept at reflux for 18 hr. The solid residue formed was filtered off and dried to obtain 9.5 g (78%) of the desired crude product (24) which was used without further purification. ¹H-NMR (DMSO-d₆) δ: 8.40(s, 1H), 8.30(d, 1H), 8.20(d, 1H), 8.10(d, 1H), 7.60(s, 1H), 7.35(d, 1H), 6.45(m, 12H), 3.30(m, 16H), 1.10(m, 24H). Ion spray mass spectrometry: calculated for C₂₉H₃₀N₂O₅ 486 Da, found 487 Da.

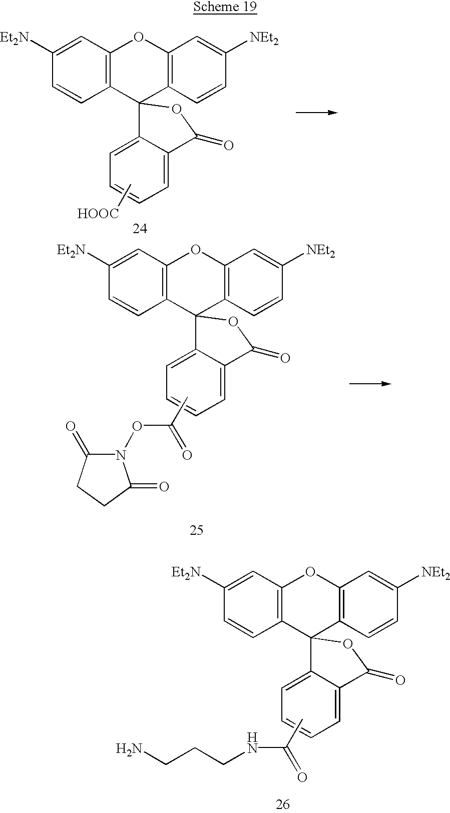

Synthesis of 5(6) Carboxytetraethylrhodamine N-Succinimidyl Ester (2.5):

A solution of acid (24) (1.00 g, 2.00 mmol) and N-hydroxysuccinimide (260 mg, 2.20 mmol) in 20 mL DMF was stirred at room temperature while a solution of DCC (470 mg, 2.20 mmol) in 220 mL DMF was added dropwise. At the end of the reaction (TLC control, silica, CH₃CN/water 8:2) solvents were distilled off in vacuo and the oily residue was purified by chromatography (silica, CH₃CN/water 9:1) to obtain 437 mg of product (25) (37%). ¹H-NMR (DMSO-d₆) δ: 8.50(s, 1H), 8.40(d, 1H), 8.35(d, 1H), 8.20(d, 1H), 7.35(s, 1H), 7.05(d, 1H), 6.60(m, 4H), 5.95(m, 8H), 3.35(m, 16H), 2.90(m, 8H), 1.12(m, 24H). Ion spray mass spectrometry: calculated for C₃₃H₃₃N₃O₇ 583 Da, found 584 Da.

Synthesis of 5(6)-(1,3-diaminopropane-carboxamiido)-tetraethyl-rhodamine (26):

A solution of the activated ester (25) (400 mg, 0.68 mmol) in 4 mL DMF was added dropwise to a solution of 1,3-diaminopropane (1.2 mL, 14 mmol) in 12 mL DMF. At the end of the reaction (silica, CH₃CN/water 8/2) solvents were distilled off in vacuo and the oily residue was treated with TFA, concentrated again and purified by chromatography (RP C18, CH₃CN/water 95:5 B90:10-80:20 0.1% TFA) to obtain 114 mg of the TFA salt (26) (34%). Ion spray mass spectrometry: calculated for C₃₂H₃₈N₄O₄ 543 Da, found 544 Da.

Synthesis of 3-Maleimido Propionic Acid Amide of 5(6)-(1,3-diaminopropanecarboxamido)-tetraethylrhodamine (29):

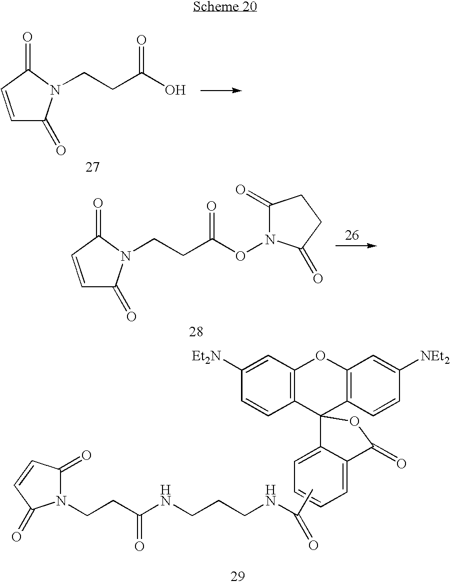

A solution of 3-maleimidopropionic acid (100 mg, 97%, 0.57 mmol), N-hydroxysuccinimide (79 mg, 0.68 mmol) and DCC (142 mg, 0.57 mmol) in dichloromethane (5 mL) was stirred at room temperature for 1 hr, then filtered. A solution of amine (26) (110 mg, 0.17 mmol), DIEA (28 μL, 0.17 mmol) in DMF (5 mL) was added and the resulting mixture was stirred overnight. Solvents were distilled off in vacuo and the residue was purified by prep HPLC to obtain 5 mg (4%) of product (29). ¹H-NMR (DMSO-d₆) δ: 8.70(t, 2H), 8.65(s, 1H), 8.25(d, 1H), 8.20(d, 1H), 8.00(dt, 2H), 7.85(s, 1H), 7.60(d, 1H), 7.00(m, 16H), 3.45(m, 20H), 3.10(m, 8H), 2.30(m, 4H), 1.15(m, 4H), 1.20(m, 24H). Ion spray mass spectrometry: calculated for C₃₉H₄₃N₅O₇ 693 Da, found 694 Da.

Synthesis of a Fluorescein Derivative Suitable for Cleavage of Polyamides from Ellman Safety-Catch Linker:

6-(3,3′-diamino-N-methyldipropylamine)carboxamide fluorescein (31):

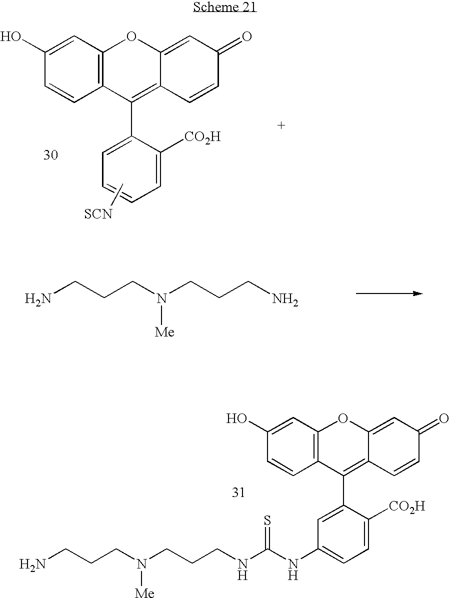

A solution of 3,3′-diamino-N-methyldipropylamine (0.4 mL, 96%, 2.5 mmol) in 20 mL dichloromethane was cooled in an ice bath. A solution of fluorescein isothiocyanate (100 mg, 0.26 mmol) in 20 mL MeOH was added dropwise. The resulting mixture was stirred 2 hr, filtered and concentrated in vacuo. Chromatographic purification of the residue (RPC18, CH₃CN/water 5:95, 0.1% TFA) afforded 20 mg of pure isomer (31) as the TFA salt. Ion spray mass spectrometry: calculated (average isotopic composition) 534 Da, found 535 Da.

Cleavage of the Polyamide from the Resin with Aminoderivatives of Fluorescein (32):

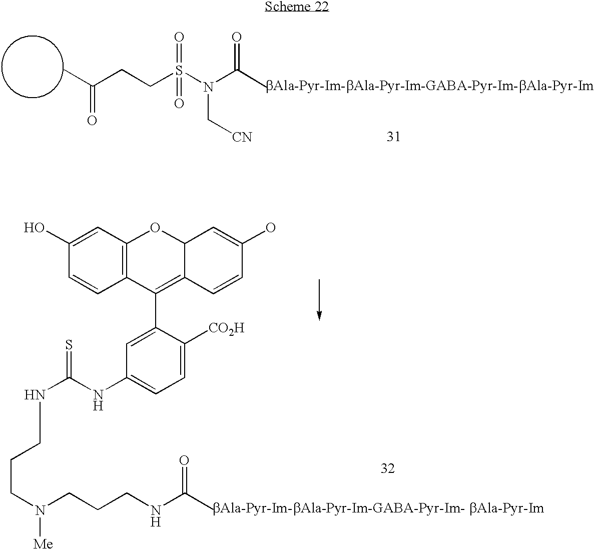

A batch of resin synthesized and alkylated as previously described (77 mg, 9.6 μmol) was swelled in DMF and treated with a solution of (31) (15 mg, 19 μmol) and DIEA (14 μL, 77 μmol) in 1 mL DMF shaking for 12 hr. Filtration and concentration left 40 mg of an oil. Preparative HPLC purification afforded 2.5 mg (14%) of product (32). ¹H-NMR (DMSO-d₆) δ: 8.45(s, 1H), 8.20(d, 1H), 7.90(d, 1H), 7.70(m, 1H), 7.20(d, 1H), 6.60(m, 6H), 3.60(m, 2H), 3.20(m, 4H), 2.85(m, 2H), 2.80(s, 3H), 1.90(m, 4H). Ion spray mass spectrometry: calculated for C₈₇H₉₆F₃N₂₇O₁₉S (average isotopic composition) 1798 Da, found 1799 Da.

Ligation of Polyamide Thiols with Maleimido Derivatives of Rhodamine

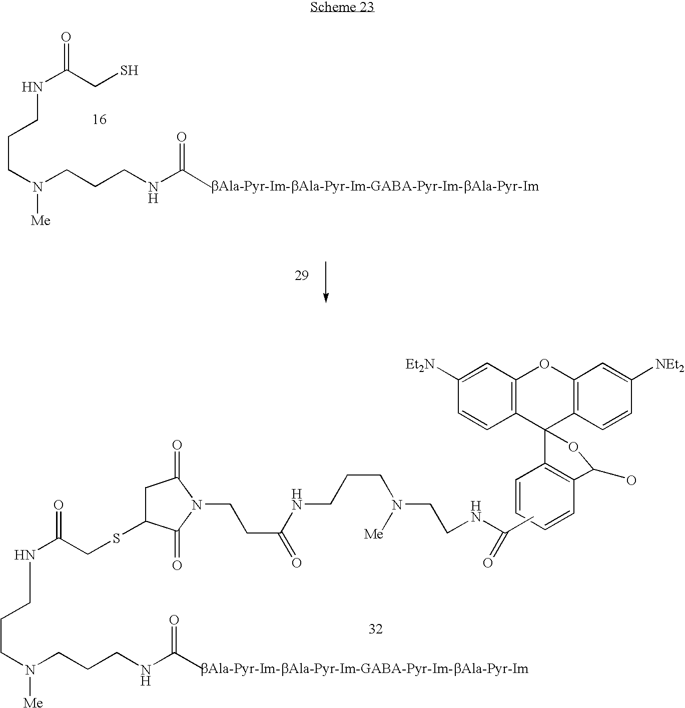

Thiol (16) (2.3 mg, 1.4 μmol) and maleimido derivative (29) were dissolved in 0.5 mL DMF together with 20 mL (116 μmol) of DIEA and shaken at room temperature for 12 hr. Solvents were then distilled off in vacuo and the residual red oil purified by preparative HPLC to obtain 0.5 mg of (33) (17%). Ion spray mass spectrometry: calculated for C₁₀₅H₁₂₉N₃₁O₂S (average isotopic composition) 2177 Da, found 2177 Da.

### Example 13

### Synthesis of Rhodamine Labelled Peptide-Polyamide Conjugates

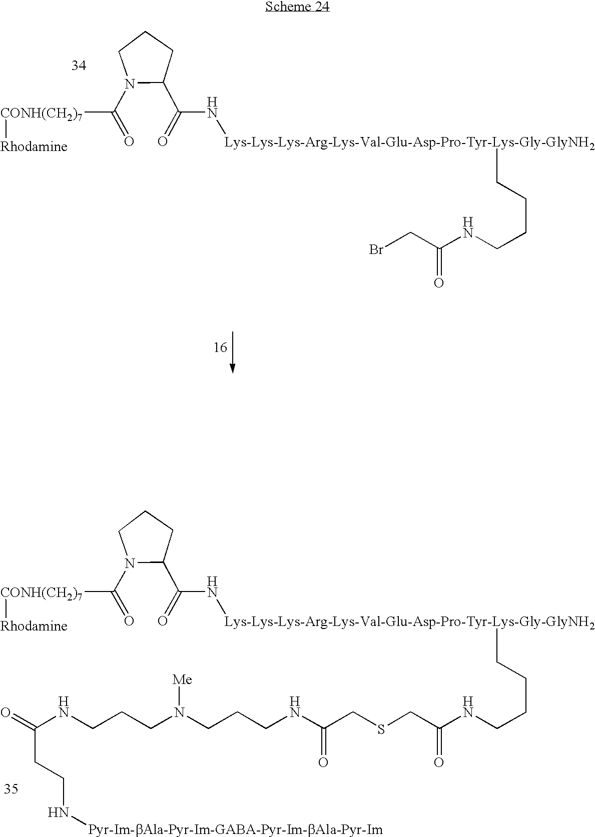

Synthesis of Rhodamine Labelled Bromoacetyl-NLS Peptide: Rhodamine-Aoc-Pro-Lys-Lys-Lys-Arg-Lys-Val-Gluu-Asp-Pro-Tyr-Lys(Br—CH₂—CO)-Gly-Gly-CO—NH₂ (34):

Peptide Assembly:

The peptide was synthesized as described for (17) except the N-terminal Pro (NLS sequence) was incorporated as the Fmoc derivative, and Aoc (aminooctanoic acid) as Fmoc-Aoc-OH. Rhodamine (24) (1 eq) was pre-activated with DIPC (1 eq) and HOBt (1 eq), and coupled on solid phase for 1 hr, using a 5-fold excess over the resin amino groups.

Cleavage of N^{ε}-Allyloxycarbonyl Protecting Group of the C-Terminal Lysine:

As described for (17).

Coupling of Bromoacetic Acid:

As described for (17).

Cleavage of Rhodamine-NLS-Bromoacetylpeptide (34) from the Resin:

As described for (17).

Purification of Rhodamine-NLS-Bromoacetylpeptide (34):

The peptide was purified by preparative HPLC on a Waters Delta-Pak C-18 column (20×200 mm). In a typical run, the crude peptide (18 mg) was dissolved in water, 0.1% TFA, loaded onto the preparative column and eluted with a linear gradient between 20%-35% in 20 min at a flow rate of 30 mL/min. The fractions containing the desired peptide (>98% pure) were pooled and lyophilized, yield 4 mg (22%). Ion-spray mass spectrometry of the HPLC purified peptide gave the expected molecular weight: calculated (average isotopic composition) 2359 Da, found 2359.2 Da.

### Example 14

### Synthesis of the Peptide/Polyamide Conjugates (35)

The rhodamine labelled bromoacetyl-NLS peptide (34) (1.1 mg) and (16) (0.72 mg) were dissolved in 200 μL of DMF and 2 μL DIEA. The reaction was monitored by analytical HPLC. After 30 min the reaction was complete and the solution was immediately purified by HPLC, using a linear gradient between 25%-40% of B in 30 min, with a flow rate of 1 mL/min. The fractions containing the desired product were collected and freeze-dried, yielding 0.2 mg (11%) of (35). Ion-spray mass spectrometry gave the expected molecular weight: calculated (average isotopic composition) 3762 Da, found 3762.3 Da.

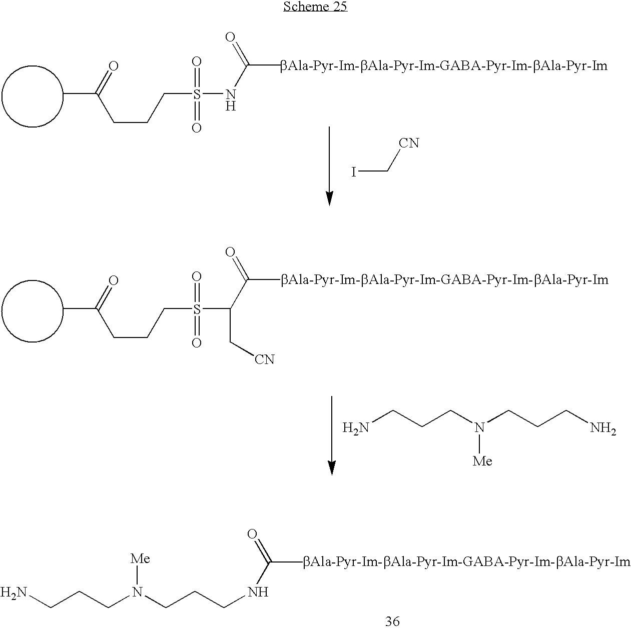

A batch of resin containing the sequence. (Resin)-βAla-Pyr-Im-βAla-Pyr-Im-GABA-Pyr-Im-βAla-Pyr-Im, synthesized according to the above procedure (54 mg, 7 μmol th.) was treated with 0.25 mL (4.5 mmol) iodoacetonitrile and 0.14 mL (0.8 mmol) DIEA in 0.6 mL NMP for 4 hr, then washed with NMP, DCM and dried under a stream of nitrogen. A solution of 3,3′-diamino-N-methyl-dipropylamine (50 μL, 280 μmol) in 0.5 mL DMF was then added and the resin stirred overnight at room temperature. The solution was filtered and concentrated in vacuo. The residue was triturated with diethylether to obtain 33 mg of a yellow oil.

The crude polyamide was purified by preparative HPLC: column RP-C18, 100×19 mm, 5 μm, flow rate 20 mL/min, eluent A, water (0.1% TFA); eluent B acetonitrile (0.1% TFA), sample loaded in 0.8 mL neat DMF at 98% eluent A, then linear gradient from 2% to 60% eluent B in 40 min. Fractions containing the desired compound were pooled and lyophilized, yielding 4.8 mg of desired product (36) (42%). The purified compound was analyzed by ion spray mass spectrometry and gave the expected molecular weight: calculated (average isotopic composition) 1409.6 Da, found 1409.

### Example 16

### Derivatization of Polyamide (36) with 5-Oxohexanoic Acid

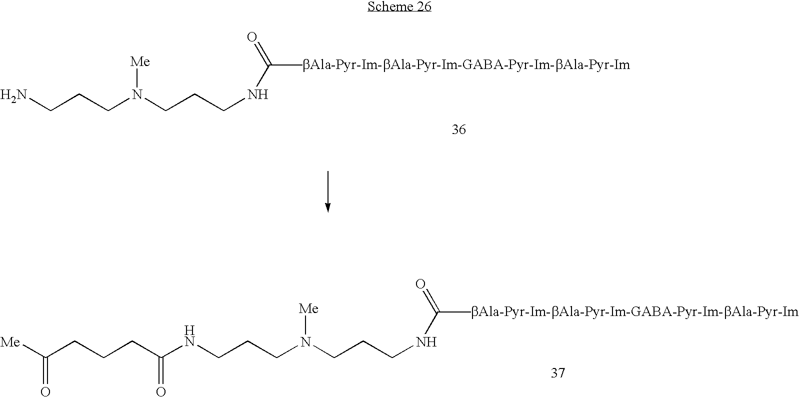

Polyamide (36) (4.8 mg 2.9 μmol), 5-oxo-hexanoic acid N-hydroxysuccinimide ester (20 mg, 88 μmol) and 25 μL DIEA were dissolved in 50 μL DMF. After 25 min, 100 μL DMF and 12 μL acetic acid were added. The solution was filtered and the product (37) was purified in three portions by semi-preparative HPLC: column RP-C18, 150×7.8 mm, 7 μm, flow rate 3.6 mL/min, eluent A, water (0.1% TFA), eluent B acetonitrile (0.1% TFA), linear gradient from 5% to 60% eluent B in 30 min. Fractions containing the desired compound were pooled and lyophilized, yielding 2.2 mg of desired product (37) (46%). The purified compound was analyzed by ion spray mass spectrometry and gave the expected molecular weight: calculated (average isotopic composition) 1521.7 Da, found 1521 Da.

### Example 17

### Synthesis of Mannoside Cluster (42)

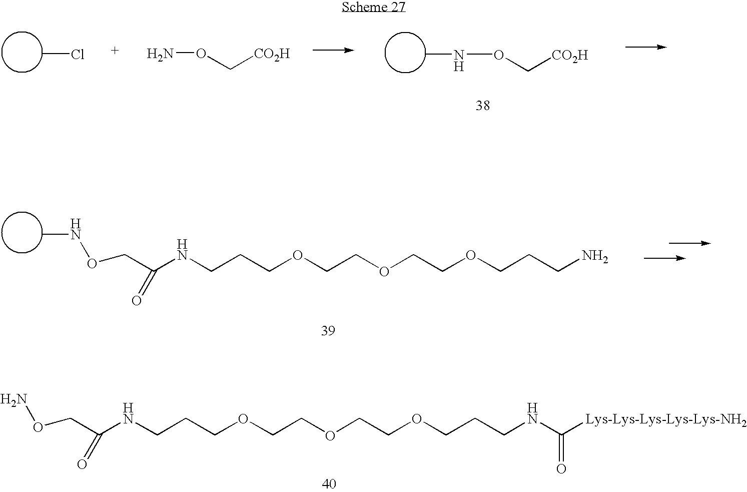

The procedure followed the one described in Liebigs Ann. Chem., 1079 (1988). Briefly, to 8 mL of a 5:1 mixture of anhydrous chloroform and anhydrous acetonitrile were added carboxy-methoxylamine hemihydrochloride (550 mg, 2.6 mmol), thriethyl-amine (180 μL, 0.5 eq) and trimethylchlorosilane (330 μL, 2.6 mmol). The resulting white suspension was heated to reflux for 2 hr. The suspension was cooled to 0° C., before triethylamine (600 μL, 4.3 mmol, 1.7 eq) was added. After stirring for 10 min at room temperature 4-polystyryltri-phenylmethylchloride (PS-trityl chloride) (1 g, 1.24 mmol) of resin was added. The mixture was slowly magnetically stirred at room temperature for 6 hr. The resin was then transferred into a syringe and sequentially washed with chloroform, methanol, 5% aq. citric acid, water, methanol, THF and DCM. A negative Kaiser test indicated the absence of free primary amino groups. The resin (38) was dried for 1 hr under high vacuum.

Resin (38) was further evaluated by a coupling with β-alanine-fluorenylmethylester: Resin (38) (25.65 mg) was swollen for 30 min. in 300 μL DMF, drained and carbonyldiimidazole (63 mg, 0.39 mmol), dissolved in 300 μL DMF was added. The resin was shaken for 50 min, drained and washed with DMF. Then β-alanine-fluorenylmethylester (TFA salt) (38.0 mg, 0.099 mmol), HOBt (13.2 mg, 0.086 mmol) and DIEA (37 μL, 2.4 eq resp. amino acid), dissolved in 300 μL DMF were added. The resin was shaken for 1 hr, drained and washed with DMF and DCM. After 1 hr under high vacuum, the loading was determined by Fmoc quantitation and found to be 0.337 mmol/g.

Resin (39):

The procedure followed the one described in J. Am. Chem. Soc., Vol. 121, 7034 (1999). Briefly, resin (39) was pre-swollen in DMF, drained and a solution of carbonyldiimidazole (1.3 g) in 8 mL DMF was added. The resin was shaken for 30 min, drained and washed with DMF. Then a 0.5 M solution of HOBt in 8 mL of a 1:1 (v/v) mixture of DMF and 4,7,10-trioxa-1,13-tridecane-diamine was added. After initial short vortexing, the resin was shaken for 1 hr, then drained and washed with DMF and DCM. The Kaiser test showed an intense blue colour, indicative of free primary amino groups. The resin (39) was dried under high vacuum for 1 hr (dry weight: 1.27 g).

Peptide (40):

The synthesis of the polylysine peptide moiety was continued by standard Fmoc chemistry. Coupling: (β-Fmoc-L-Lys(ε-Boc)-OH amino acid, PyBOP, HOBt and DIEA (molar ratios 1:1:1:2) were used with three fold molar excess with respect to resin loading (coupling time 1 hr). Each coupling step was controlled by the Kaiser test. Deprotection: The resin was treated with 20% piperidine in DMF for 20 min. The loading of the resin was determined by Fmoc-quantitation after the attachment of the first lysine residue and found to be 0.224 mmol/g (dry weight 1.417 g). Cleavage/Boc-deprotection: The resin was treated with reagent B (1.25 g phenol/1.25 mL water/0.5 mL TIPS dissolved in 25 mL TFA) for 2 hr. The deprotected peptide (40) was precipitated with cold MTBE and purified by preparative HPLC: column RP-C18, 150×19 mm, 5 μm, flow rate 20 mL/min, eluent A, water (0.1% TFA), eluent B acetonitrile (0.1% TFA), linear gradient from 2% to 30% eluent B in 40 min. Fractions containing the desired compound were pooled and lyophilized, yielding (40) (375 mg, 0.216 mmol, MW calculated with 7×TFA, 68% yield respective the loading determined after the first coupling). The purified compound was analyzed by ion spray mass spectrometry and gave the expected molecular weight: calculated (average isotopic composition) 934.2 Da, found 934.

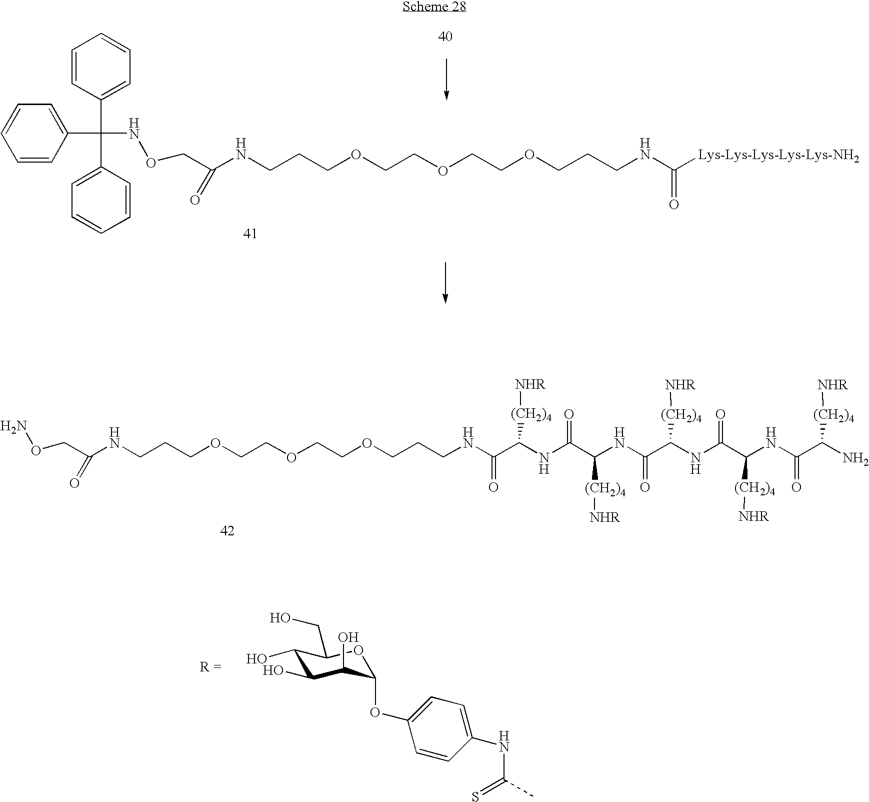

Protected Peptide (41):

Peptide (40) (188 mg, 0.107 mmol, MW calc. with 7×TFA) was dissolved in 5 mL acetic acid. Then a solution of triphenyl-methylcarbinol (93 mg, 0.36 mmol) in 2 mL DCM was added. To the resulting clear solution was added dropwise under stirring BF₃.OEt₂ (44 μL, 0.36 mmol). The mixture was stirred for 100 min at room temperature. The reaction mixture was injected into cold diethyl ether. The precipitate was dissolved in water, filtered and purified by preparative RP-HPLC: column RP-C18, 150×19 mm, 5 μm, flow rate 20 mL/min, eluent A, water (0.1% TFA), eluent B acetonitrile (0.1% TFA), linear gradient from 2% to 30% eluent B in 40 min. Fractions containing the desired compound were pooled and lyophilized, yielding (41) (160 mg, 0.086 mmol, MW calc. with 6×TFA, 80%). The purified compound was analyzed by ion spray mass spectrometry and gave the expected molecular weight: calculated (average isotopic composition) 1176.6 Da, found 1177.

Mannoside Cluster (42):

To a solution of (41) (63 mg, 35 μmol) in a mixture of 2 mL DMF and 2 mL 0.1 M aqueous NaHCO₃ was added 1-O-(4-isothiocyanato-phenyl)-α-D-mannopyranose (79 mg, 250 μmol). The reaction progress was monitored by analytical HPLC. After 18 h further 1-O-(4-isothiocyanato-phenyl)-α-D-mannopyranose (54 mg) and NaHCO₃ (20 mg) were added. After 22 h the reaction was complete. To the mixture were added 10 mL water. The resulting white precipitate was filtered off. The precipitate was suspended in 2 mL water and 100 μL of isopropylamine were added. After 10 min stirring at room temperature 2 mL ethanol were added and the mixture was evaporated to dryness in vacuo. The dry precipitate was triturated with diethylether and left under high vacuum for 2 hours.

Deprotection:

The crude reaction product was deprotected by dissolving it in a mixture of 20 mL DCM, 400 μL TIPS and 600 μL TFA. A yellow, clear solution resulted, which was stirred for 10 min. at room temperature, before 200 μL of water were added. The solution was then poured into 100 mL MTBE and the resulting precipitate was washed with 50 mL MTBE. The precipitate was lyophilized to afford 66.3 mg of crude. The deprotected manno-side cluster (42) was purified by preparative RP-HPLC: column RP-C18, 150×19 mm, 5 μm, flow rate 20 mL/min, eluent A, water (without TFA), eluent B acetonitrile (without TFA), linear gradient from 3% to 45% eluent B in 40 min. Fractions containing the desired compound were pooled and lyophilized, yielding (42) (29.3 mg, 29.7%, calc. from 41). The purified compound was analyzed by ion spray mass spectrometry and gave the expected molecular weight: calculated (average isotopic composition) 2814.2 Da, found 2814.

### Example 18

### Synthesis of Mannoside Cluster/Polyamide Conjugate (43)

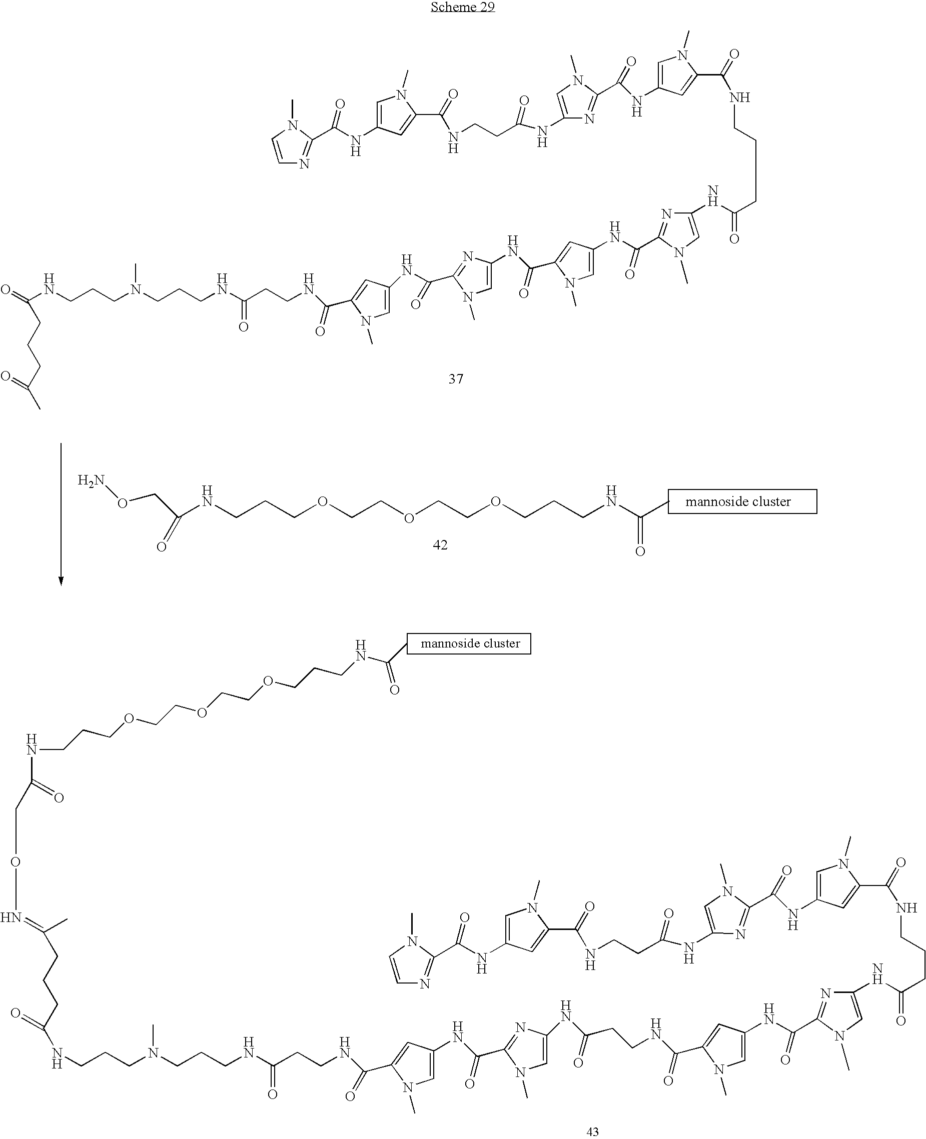

Polyamide (37) (1.1 mg, 0.72 μmol) and mannoside cluster (42) (7.0 mg, 2.3 μmol) were dissolved in a mixture of 50 μL DMF and 30 μL 0.1 M aqueous sodium acetate buffer, pH 4.0 The solution was left standing at room temperature for 70 min. The conjugate (43) was purified by semipreparative RP-HPLC: column RP-C18, 150×7.8 mm, 7 μm, flow rate 3.6 mL/min, eluent A, water (without TFA), eluent B acetonitrile (without TFA), linear gradient from 5% to 60% eluent B in 30 min. Fractions containing the desired compound were pooled and lyophilized, yielding (43) (0.29 mg, 9.3%). The purified compound was analyzed by ion spray mass spectrometry and gave the expected molecular weight: calculated (average isotopic composition) 4317.9 Da, found 4318.

### Example 19

### Characterization of Peptide-Polyamide DNA Conjugates

The ability of peptide-polyamide conjugates to bind the DNA was assessed by circular dichroism (CD) spectroscopy and by gel electrophoresis.

CD spectropolarimetry provides a means for detecting the binding of the hairpin polyamide to the target DNA binding site as a oligoduplex as reported in Pilch, D. S., et al., Biochemistry, Vol. 38, 2143-2151, 1999. CD spectra are recorded from 220 to 380 nm by incremental titration of the polyamide or peptide-polyamide into a solution of the target twelve-mer duplex.

A 5 μM solution of both the complementary oligonucleotides ACATGCAGCTCCC and GGGAGCTGCATGTT was prepared in sodium cacodylate 10 mM, KCl 10 mM, MgCl₂ 10 mM, CaCl₂ 5 mM. The solution was boiled in an Eppendorf thermomixer, at 95□C. for 5 min, for annealing.

A 0.55 mM stock solution of (18) was prepared by dissolving 0.56 mg of freeze-dried product in 200 mL of water. CD spectra in the 220-380 region were recorded of the solutions obtained by incremental titration of (18) into the solution of oligoduplex at 20° C. Upon addition of (18), a substantial CD signal arises at 315 nm which was indicative of binding. Moreover it is possible from the titration curve to derive the estimate of a ligand-duplex stoichiometry of 1.

Neither the polyamide nor the oligoduplex exhibit CD signals in the polyamide absorbing 300-380 nm wavelength region. Thus the induced CD signal is indicative of interactions between the polyamide and the DNA.

### Example 20

### Binding of Peptide-Polyamide Conjugate to Plasmid DNA

The ability of the polyamides to bind the DNA plasmid was assessed by gel electrophoresis of complexes of plasmid and fluorescently labelled polyamides.

A plasmid pCMV/mEPO was constructed by inserting the mouse EPO (mEPO) coding sequence as a EcoRI-BamHI 0.6 Kb fragment into pViJnsB (Montgomery, D. L., Shiver J. W., Leander K. R., Perry, H. C., Friedman, A., Martinez, D., Ulmer, J. B., Donnelly, J. J., Liu, M. A. (1993) DNA Cell. Biol. 12, 777-783.), which contains the CMV immediate/early region promoter and enhancer with intron A followed by the BGH polyadenylation signal. The mouse EPO coding region including 40 bp of the 5′ untranslated region (Shoemaker, C. B., & Mitsock, L. D. (1986) Mol. Cell. Biol. 6, 849-858) was assembled from synthetic oligonucleotides as described (Stemmer, W. P. C, Crameri, A., Ha, K. D., Brennan, T. M., & Heyneker, H. L. (1995) Gene 164, 49-53.).

In a typical experiment either the rhodamine labelled polyamide (33), the peptide-polyamide (35) and the correspondent rhodamine-peptide as control, were dissolved in DMSO at a 0.55 mM concentration. Serial dilutions of the polyamides and the peptide were prepared in DMSO. The plasmid stock solution was 0.54 μM. The polyamide-plasmid complexes were prepared by incubating 9 mL of water, 1 μL of plasmid and 3 μL of each polyamide serial dilution solution to form a 100×, 10×, 7×, 5×, 3×, 1× of polyamide with respect of plasmid concentration. After 10 min or 5 hr incubation, the solutions were loaded on a agarose gel. The fluorescent label of polyamides co-migrates with the band correspondent to the plasmid at a ratio polyamide/plasmid>5. The control rhodamine-peptide (34) does not co-migrate with the plasmid.

### REFERENCES

A number of patents and publications are cited herein in order to more fully describe and disclose the invention and the state of the art to which the invention pertains. Each of these references is incorporated herein by reference in its entirety into the present disclosure.

Branden, L. J., Mohamed, A. J. and Smith, C. I. E. Nat. Biotechnol. 17 (1999) 784-787.

Bremer, R. E., Baird, E. E. and Dervan, P. B. Chem Biol. 5 (1998) 119-133.

Ciolina, C., Byk, G., Blanche, F., Thuillier, V., Scherman, D. and Wils, P. Bioconj. Chem. 10 (1999) 49-55.

Collas, P. and Alestrom, P., Mol. Repr. Dev. 45 (1996) 431-438.

Cooper, R. G., Harbottle, R. P., Schneider, H., Coutelle, C. and Miller, A. D. Angew. Che. Int. Ed. 38 (1999) 1949-1952. Peptide Mini-Vectors for Gene Delivery.

Dickinson, L. A., Gulizia, R. J., Trauger, J. W., Baird, E. E., Mosier, D. E., Gottesfeld, J. M. and Dervan, P. B. Proc. Natl. Acad. Sci. U.S.A. 95 (1998) 12890-12895.

Ding, Z., Cristiano, R. J., Roth, J. A., Takacs, B. and Kuo, M. T. J. Biol. Chem. 270 (1995) 3667-3676.

Ferkol, T., Perales, J. C., Mularo, F. and Hanson, R. W., Proc. Natl. Acad. Sci. U.S.A. 93 (1996) 101-105.

Gottesfeld, J. M., Neely, L., Trauger, J. W., Baird, E. E. and Dervan, P. B. Nature 387 (1997) 202-205.

Harbottle, R. P., Cooper, R. G., Hart, S. L., Ladhoff, A., McKay, T., Knight, A. M., Wagner, E., Miller, A D. and Coutelle, C. Human gene Ther. 9 (1998) 1037-1047.

Muir, T. W., Williams, M. J., Ginsberg, M. H. and Kent, S. B. H. Biochemistry 33 (1994) 7701-7708.

Neves, C., Byk, G., Scherman, D. and Wils, P. FEBS Lett. 453 (1999) 41-45.

Sebestyen, M. G., Ludtke, J. J., Bassik, M. C., Zhang, G., Budker, V., Lukhtanov, E., Hagstrom, J. E. and Wolff, J. A. Nat. Biotechnol. 16 (1998) 80-85.

Smith, R. M. and Wu, G. Y., Sem. Liver Dis. 19 (1999) 83-92.

Sobolev, A. S., Rosenkranz, A. A., Smirnova, O. A., Nikitin, V. A., Neugodova, G. L., Naroditsky, B. S., Shilov, I. N. and Ernst, L. K., J. Biol. Chem. 273 (1998) 7928-7933.

Trauger, J. W., Baird, E. E. and Dervan, P. B. J. Am. Chem. Soc. 120 (1998) 3534-3535.

Wagner, E., Zenke, M., Cotton, M., Beug, H. and Birnstiel, M., Proc. Natl. Acad. Sci. U.S.A. 87 (1990) 3410-3414.

Wemmer, d. E. and Dervan, P. B. Curr. Opin. Struc. Biol. 7 (1997) 355-361.

Zanta, M. A., Belguise-Valladier, P. and Behr, J. P. Proc. Natl. Acad. Sci. U.S.A. 96 (1999) 91-96.

Zelphati, O., Liang, X., Hobart, P. and Felgner, P. L., Human Gene Ther. 10 (1999) 15-24.

1. A vector comprising:
(a) a double stranded DNA (dsDNA) having at least one target sequence; and,
(b) a chimeric molecule comprising:
(i) a sequence specific polyamide (SSP) moiety bound non-covalently to said target sequence; and,
(ii) a ligand moiety linked covalently to said sequence specific polyamide.

2. A vector according to claim 1, wherein said double stranded DNA has more than one target sequence.

3. A vector according to claim 1, wherein said double stranded DNA has from 2 to 100 target sequences.

4. A vector according to claim 1, wherein said double stranded DNA has from 2 to 10 target sequences.

5. A vector according to claim 1, wherein said double stranded DNA has from 3 to 10 target sequences.

6. A vector according to claim 1, wherein said double stranded DNA has from 4 to 10 target sequences.

7. A vector according to claim 1, wherein said double stranded DNA has from 5 to 10 target sequences.

8. A vector according to claim 1, wherein said double stranded DNA has from 6 to 10 target sequences.

9. A vector according to any one of claims 1-8, wherein said target sequence is not present in the promoter or coding sequence part of the vector.

10. A vector according to any one of claims 1-9, wherein said target sequence is at least 6 bases in length.

11. A vector according to any one of claims 1-9, wherein said target sequence is at least 8 bases in length.

12. A vector according to any one of claims 1-9, wherein said target sequence is 6-20 bases in length.

13. A vector according to any one of claims 1-9, wherein said target sequence is 8-20 bases in length.

14. A vector according to any one of claims 1-9, wherein said target sequence is 10-20 bases in length.

15. A vector according to any one of claims 1-14, wherein said double stranded DNA further comprises dsDNA sequences of human, non-human animal, vegetable, bacterial, or viral origin.

16. A vector according to any one of claims 1-15, wherein said double stranded DNA further comprises one or more of transcribable sequences, promoters, and origins of replication.

17. A vector according to any one of claims 1-15, wherein said double stranded DNA further comprises one or more selectable or detectable markers.

18. A vector according to any one of claims 1-15, wherein said double stranded DNA further comprises one or more sequences designed to facilitate homologous recombination to a specific locus within a host cell.

19. A vector according to any one of claims 1-18, wherein said double stranded DNA is linear.

20. A vector according to any one of claims 1-18, wherein said double stranded DNA is circular.

21. A vector according to any one of claims 1-18, wherein said double stranded DNA is circular supercoiled DNA.

22. A vector according to any one of claims 1-21, wherein said-double stranded DNA further comprises a transcribable sequence which, when transcribed from the DNA under the control of a promoter, brings about a therapeutic effect.

23. A vector according to any one of claims 1-21, wherein said double stranded DNA further comprises a transcribable sequence which, when transcribed from the DNA under the control of a promoter, yields mRNA for the expression of a protein.

24. A vector according to any one of claims 1-21, wherein said double stranded DNA further comprises a transcribable sequence which, when transcribed from the DNA under the control of a promoter, yields RNA which itself has a function as an anti-sense RNA or a ribozyme.

25. A vector according to any one of claims 1-21, wherein said double stranded DNA further comprises one or more coding sequences designed to modify tumour cells so that the tumour cells may be destroyed or inactivated.

26. A vector according to any one of claims 1-21, wherein said double stranded DNA further comprises one or more genes encoding enzymes capable of activating pro-drugs into active toxic drugs.

27. A vector according to any one of claims 1-21, wherein said double stranded DNA further comprises one or more tumour suppressor genes.

28. A vector according to any one of claims 1-21, wherein said double stranded DNA further comprises one or more genes encoding cytokines or cell surface markers of the immunoglobulin superfamily.

29. A vector according to any one of claims 1-21, wherein said double stranded DNA further comprises one or more functional copies of a gene.

30. A vector according to any one of claims 1-21, wherein said double stranded DNA further comprises DNA encoding antigens useful as vaccines.

31. A vector according to any one of claims 1-21, wherein said double stranded DNA further comprises one or more of constitutive promoters and tissue specific promoters.

32. A vector according to any one of claims 1-31, wherein said sequence specific polyamide moiety comprises at least 6 organic heterocyclic groups, at least some of which are pyrrole and imidazole groups.

33. A vector according to any one of claims 1-32, wherein said sequence specific polyamide moiety comprises at least 7 organic heterocyclic groups.

34. A vector according to any one of claims 1-32, wherein said sequence specific polyamide moiety comprises 8 or more organic cyclic groups.

35. A vector according to any one of claims 1-35, wherein said sequence specific polyamide moiety comprises not more than about 30 organic cyclic groups.

36. A vector according to any one of claims 1-35, wherein said sequence specific polyamide moiety comprises not more than about 20 organic cyclic groups.

37. A vector according to any one of claims 1-35, wherein said sequence specific polyamide moiety comprises not more than about 18 organic cyclic groups.

38. A vector according to any one of claims 1-37, wherein at least 60% of said organic cyclic groups are organic heterocyclic groups.

39. A vector according to any one of claims 1-37, wherein at least 80% of said organic cyclic groups are organic heterocyclic groups.

40. A vector according to any one of claims 1-37, wherein 100% of said organic cyclic groups are organic heterocyclic groups.

41. A vector according to any one of claims 1-40, wherein said organic heterocyclic groups have five or six annular members.

42. A vector according to any one of claims 1-40, wherein said organic heterocyclic groups have five annular members.

43. A vector according to any one of claims 1-40, wherein said organic heterocyclic groups have from 1 to 3 annular heteroatoms selected from nitrogen, oxygen, and sulfur.

44. A vector according to any one of claims 1-40, wherein said organic heterocyclic groups have from 1 to 2 annular heteroatoms selected from nitrogen, oxygen, and sulfur.

45. A vector according to any one of claims 1-40, wherein said organic heterocyclic groups have from 1 to 3 annular nitrogen heteroatoms.

46. A vector according to any one of claims 1-40, wherein said organic heterocyclic groups have from 1 to 2 annular nitrogen heteroatoms.

47. A vector according to any one of claims 1-40, wherein said organic heterocyclic groups are selected from optionally substituted pyrrole, imidazole, pyrazole, triazole, furan, thiophene, oxazole, thiazole, and cyclopentadiene.

48. A vector according to any one of claims 1-40, wherein said organic heterocyclic groups are selected from optionally substituted pyridine, pyrimidine, and triazine.

49. A vector according to any one of claims 1-48, wherein one or more annular NH groups are substituted with C₁₋₃alkyl groups.

50. A vector according to any one of claims 1-48, wherein one or more annular NH groups are substituted with methyl.

51. A vector according to any one of claims 1-50, wherein said organic heterocyclic groups have five annular members, 1 to 2 annular nitrogen atoms, and one annular nitrogen atom which is methylated.

52. A vector according to any one of claims 1-51, wherein said organic heterocyclic groups are selected from N-methyl pyrrole (“Py”) and N-methyl imidazole (“Im”) units.

53. A vector according to any one of claims 1-52, wherein said sequence specific polyamide moiety further comprises one or more optionally substituted aliphatic amino acid groups having a chain of two to six carbon atoms.

54. A vector according to any one of claims 1-52, wherein said sequence specific polyamide moiety further comprises two or more optionally substituted aliphatic amino acid groups having a chain of two to six carbon atoms.

55. A vector according to any one of claims 1-54, wherein said sequence specific polyamide moiety further comprises no more than 6 optionally substituted aliphatic amino acid groups.

56. A vector according to any one of claims 1-54, wherein said sequence specific polyamide moiety further comprises no more than 4 optionally substituted aliphatic amino acid groups.

57. A vector according to any one of claims 1-56, wherein said sequence specific polyamide moiety further comprises an optionally substituted aliphatic amino acid groups having a chain of two to six carbon atoms proximal to at least one terminus of said moiety.

58. A vector according to any one of claims 1-57, wherein said optionally substituted aliphatic amino acid groups are selected from glycine, beta-alanine, and gamma-aminobutyric acid.

59. A vector according to any one of claims 1-58, wherein no consecutive sequence of 6 heterocycles is present.

60. A vector according to any one of claims 1-59, wherein said organic cyclic groups and aliphatic amino acid groups, if present, are joined by linking groups having a length of two atoms, wherein at least some of the linking groups will have NH groups.

61. A vector according to any one of claims 1-59, wherein said organic cyclic groups and aliphatic amino acid groups, if present, are joined by linking groups selected from methyleneamino (—CH₂—NH—), carboxamide (—C(═O)NH—), ethylene (—CH₂CH₂—), thiocarboxamide (—C(═S)NH—), and carboxamidinoyl (—C(═NH)NH—).

62. A vector according to any one of claims 1-59, wherein said organic cyclic groups and aliphatic amino acid groups, if present, are joined by linking groups selected from carboxamide (—C(═O)NH—), thiocarboxamide (—C(═S)NH—), and carboxamidinoyl (—C(═NH)NH—).

63. A vector according to any one of claims 1-59, wherein said organic cyclic groups and aliphatic amino acid groups, if present, are joined by carboxamido groups (—C(═O)NH—).

64. A vector according to any one of claims 1-63, wherein one or both termini of the sequence specific polyamide moeity has a polar group substituted on an alkyl group, where said polar group is from 2 to 6 carbon atoms from the linkage to the remaining molecule.

65. A vector according to claim 64, wherein said polar group is selected from amino, hydroxyl, and mercapto.

66. A vector according to claim 64, wherein said polar group is alkylated amino, where the alkyl groups are of from 1 to 6 carbon atoms, and at a pH less than about 8, the amino group is positively charged.

67. A vector according to claim 64, wherein said polar group is optionally substituted aminopropyl.

68. A vector according to claim 64, wherein said polar group is optionally substituted N-methylaminopropyl.

69. A vector according to any one of claims 1-68, wherein said sequence specific polyamide moiety has at least one complementary pair including an N-methyl imidazole group and which has specificity of one nucleotide.

70. A vector according to any one of claims 1-68, wherein said sequence specific polyamide moiety has at least two complementary pairs, each including an N-methyl imidazole group and having specificity of one nucleotide.

71. A vector according to any one of claims 1-70, wherein said ligand moiety is capable of directing the conjugate to a cellular or sub-cellular location.

72. A vector according to any one of claims 1-70, wherein wherein said ligand moiety is capable of directing the conjugate to the nucleus of a eurkaryotic cell.

73. A vector according to any one of claims 1-70, wherein wherein said ligand moiety is a general nuclear localisation signal.

74. A vector according to any one of claims 1-73, wherein wherein said ligand moiety is a protein or polypeptide capable of binding a target receptor.

75. A vector according to any one of claims 1-73, wherein said ligand moiety is a protein or polypeptide based on hormones or other protein signalling proteins which bind to a target on the surface of a cell.

76. A vector according to any one of claims 1-73, wherein said ligand moiety comprises a hybrid protein.

77. A vector according to any one of claims 1-73, wherein said ligand moiety comprises a hybrid protein including a component to direct the conjugate to a particular target cell, and a component to promote uptake of the conjugate by said cell.

78. A vector according to any one of claims 1-73, wherein said ligand moiety is selected from insulin, asialoglycoprotein or synthetic analogues thereof, transferrin, malaria circumsporozoite protein, RGD analogues, and endosomolitic peptide.

79. A vector according to any one of claims 1-73, wherein said ligand moiety is a growth factor which binds to a receptor.

80. A vector according to any one of claims 1-73, wherein said ligand moiety is an antibody or a fragment thereof.

81. A vector according to any one of claims 1-73, wherein said ligand moiety is a carbohydrate.

82. A vector according to any one of claims 1-73, wherein said ligand moiety is mannose.

83. A vector according to any one of claims 1-82, wherein said vector comprises two or more chimeric molecules which comprise different ligands moieties.

84. A composition comprising a vector according to any one of claims 1-83, and a pharmaceutically acceptable diluent or carrier.

85. A method for the synthesis of a sequence specific polyamide wherein said polyamide is synthesised on a solid support, said method comprising the steps of:
(a) attaching an N-terminal of the polyamide to the solid support via a safety-catch linker, —S(═O)₂—NH—; and,
(b) following synthesis of said polyamide, removing it from said solid support by cleavage of the safety-catch linker by activation and nucleophilic attack.

86. A method according to claim 85, wherein said safety-catch linker comprises a linkage —C(═O)—CH₂CH₂CH₂—S(═O)₂—NH—.

87. A method according to claim 85, wherein said safety-catch linker comprises a linkage —C(═O)—CH₂CH₂CH₂—S(═O)₂—NH—C(═O)—.

88. A method according to any one of claims 85-87, wherein said activation is achieved by reaction with iodoacetonitrile.

89. A method according to any one of claims 85-88, wherein said nucleophilic attack is achieved by reaction with amine or thiol.

90. A method according to any one of claims 85-89, wherein said polyamide is synthesized using one or more of the following reagents:

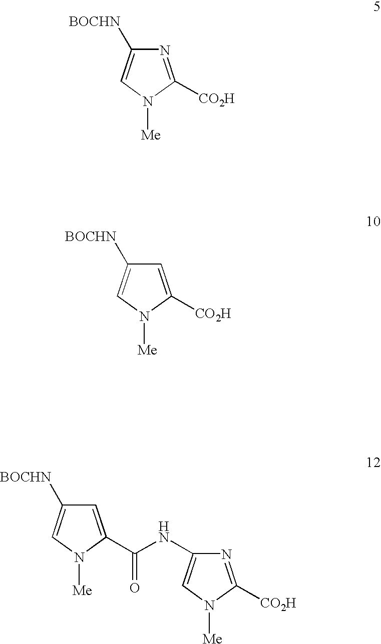

91. A method for the synthesis of a vector according to any one of claims 1-83, which includes the step of synthesis of a sequence specific polyamide by a method according to any one of claims 85-90.

92. A method of introducing a dsDNA into a cell or a sub-cellular compartment, said method comprising the steps of:
(a) providing a chimeric molecule comprising:
(i) a sequence specific polyamide moiety capable of binding non-covalently to a target nucleic acid sequence; and,
(ii) a ligand moiety linked covalently to said sequence specific polyamide moiety, and capable of being directed to said cell or said sub-cellular compartment;
(b) providing a dsDNA, said dsDNA including a target sequence for said sequence specific polyamide moiety, under conditions wherein said chimeric molecule binds to said dsDNA to provide a vector; and,
(c) bringing said vector into contact with said cell under conditions for uptake of said vector and transport of said dsDNA.

93. A method of introducing a dsDNA into the nucleus of a eukaryotic cell, said method comprising the steps of:
(a) providing a chimeric molecule comprising:
(i) a sequence specific polyamide moiety capable of binding non-covalently to a target nucleic acid sequence; and,
(ii) a ligand moiety linked covalently to said sequence specific polyamide moiety, and capable of being directed to the nucleus of said eukaryotic cell;
(b) providing a dsDNA, said dsDNA including a target sequence for said sequence specific polyamide moiety, under conditions wherein said chimeric molecule binds to said dsDNA to provide a vector; and,
(c) bringing said vector into contact with said eukaryotic cell under conditions for uptake of said vector and transport of said dsDNA.

94. A method according to claim 92 or 93, wherein step (c) is performed in vivo.

95. A method according to claim 92 or 93, wherein step (c) is performed ex vivo.

96. A method according to claim 92 or 93, wherein step (c) is performed in vitro.

97. A method according to any one of claims 92-96, wherein said chimeric molecule is as defined in any one of claims 1-83.

98. A method according to any one of claims 92-97, wherein said sequence specific polyamide moiety is as defined in any one of claims 1-83.

99. A method according to any one of claims 92-98, wherein said ligand moiety is as defined in any one of claims 1-83.

100. A method according to any one of claims 92-99, wherein said dsDNA is as defined in any one of claims 1-83.

101. A method according to any one of claims 92-100, wherein said target sequence is as defined in any one of claims 1-83.

102. A method according to any one of claims 92-101, wherein said cell is a CHO cell.

103. A eukaryotic cell obtained by a method according to any one of claims 92-102.

104. Progeny of a eukaryotic cell obtained by a method according to any one of claims 92-102.

105. A vector according to any one of claims 1-83 for use in a method of treatment of the human or animal body.

106. Use of a vector according to any one of claims 1-83 for the preparation of a medicament for the treatment of a condition treatable by gene therapy.

107. A method of gene therapy comprising administering to a patient a therapeutically effective amount of a vector according to any one of claims 1-83.
